SEQUENCE LISTING (1 sequence)

SEQ ID NO: 1 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

(19) United States
(12) Patent Application Publication
(10) Pub. No.: US 2019/0250172 A1
(43) Pub. Date: Aug. 15, 2019

(54) PROGNOSIS OF ADVERSE OUTCOMES BY DETERMINATION OF MIDKINE LEVELS AFTER CARDIOVASCULAR STRESS

(71) Applicant: CELLTREND GMBH, Luckenwalde (DE)

(72) Inventors: Harald HEIDECKE, Berlin (DE); Peter MERTENS, Magdeburg (DE)

(21) Appl. No.: 15/775,753

(22) PCT Filed: Nov. 11, 2016
(86) PCT No.: PCT/EP2016/077386

§ 371 Date: May 11, 2018

(30) Foreign Application Priority Data
Nov. 12, 2015 (EP) 15194324.8

(51) Int. Cl. G01N 33/74; A61B 5/00

(52) U.S. Cl. CPC G01N 33/74; A61B 5/4884; G01N 2800/32; G01N 2800/52; G01N 2333/475

(57) ABSTRACT

The present application relates to a method for the prognosis of adverse outcomes in a subject comprising the following steps: (i) determining the midkine level in a sample of said subject; and (ii) comparing the determined midkine level to a control midkine level; wherein said sample is taken after the subject has been subjected to cardiovascular stress; wherein said control midkine level is derived from control sample(s) from one or more subjects not showing adverse outcomes, wherein the control sample(s) have been taken after said one or more subjects not showing adverse outcomes have been subjected to cardiovascular stress; and wherein a decreased determine midkine level as compared to the control midkine level is indicative of an adverse outcome. Further it relates to a method for the prognosis of adverse outcomes in a subject comprising the following steps: (i) determining in samples of said subject the increase in midkine levels (Δmidkine value) during cardiovascular stress; and (ii) comparing the determined Δmidkine value to a control Δmidkine value; wherein said control Δmidkine value is derived from one or more subjects not showing adverse outcomes; wherein a decreased determined Δmidkine value as compared to the control Δmidkine value is indicative of an adverse outcome. It also relates to the use of a midkine antibody or an antigen binding fragment thereof for the prognosis of adverse outcomes in a subject undergoing dialysis therapy. Also encompassed is midkine for use in the treatment or the prevention of an adverse outcome, wherein midkine is administered to the subject.

### TECHNICAL FIELD OF THE INVENTION

The present invention relates to the field of medicine, in particular to the field of diagnosis and prognosis of adverse outcomes. The invention relates to the field of diagnosis and prognosis of a cardiovascular disease and mortality, such as cardiovascular mortality.

### BACKGROUND OF THE INVENTION

Midkine, a heparin binding growth factor, is involved in many biological processes, including cell survival, migration, and angiogenesis (Kadomatsu, K, and Muramatsu, T: Midkine and pleiotrophin in neural development and cancer. Cancer Lett, 204: 127-143, 2004). Midkine expression is elevated in cancer and correlates with tumor progression, due to enhanced angiogenesis (Colombo, C, et al.: Increased midkine expression correlates with desmoid tumour recurrence: a potential biomarker and therapeutic target. J Pathol, 225: 574-582, 2011; and van der Horst, E H, et al.: The growth factor Midkine antagonizes VEGF signaling in vitro and in vivo. Neoplasia, 10: 340-347, 2008). Midkine also promotes neutrophil and macrophage migration in inflammation (Sato, W, et al.: Midkine is involved in neutrophil infiltration into the tubulointerstitium in ischemic renal injury. J Immunol, 167: 3463-3469, 2001; and Kosugi, T, Sato, W: Midkine and the kidney: health and diseases. Nephrol Dial Transplant, 27: 16-21, 2012). In animal models of arterial restenosis, inflammatory cell recruitment was abrogated by midkine-deficiency and restenosis suppressed (Izumiya, et al.: Vascular endothelial growth factor blockade promotes the transition from compensatory cardiac hypertrophy to failure in response to pressure overload. Hypertension, 47: 887-893, 2006). Midkine regulates the renin-angiotensin-aldosterone system (RAAS) (Ezquerra, L, et. al: Midkine, a newly discovered regulator of the renin-angiotensin pathway in aorta: significance of the pleiotrophin/midkine developmental gene family in angiotensin II signaling. Biochem Biophys Res Commun, 333: 636-643, 2005) and participates in cross-talk between the kidney and lung. Hypertension was absent, renal damage reduced and the RAAS not activated in midkine-deficient animals (Hobo, A, et al.: The growth factor midkine regulates the renin-angiotensin system in mice. J Clin Invest, 119: 1616-1625, 2009). In this regard midkine may function as an effector molecule, linking RAAS with blood pressure regulation, fluid distribution and tissue damage.

Dialysis patients are multimorbid and suffer from diverse hormonal counter- and dysregulations (Humes, H D, Sobota, J T, Ding, F, Song, J H, Group, RADI: A selective cytopheretic inhibitory device to treat the immunological dysregulation of acute and chronic renal failure. Blood Purif, 29: 183-190, 2010). Fujisawa et al. reported increased serum midkine concentrations following application of unfractionated heparin in both dialysis patients and healthy controls.

Dialysis is known to cause cardiovascular stress (Assa S, et al. Comparison of cardiac positron emission tomography perfusion defects during stress induced by hemodialysis versus adenosine. Am J Kidney Dis. 2012; 59(6):862-864). Due to the increased loss in body fluid the cardiovascular system is stressed and therefore causes in some cases adverse outcomes like cardiac arrest. Dialysis hence represents a suited model for cardiovascular stress.

Nowadays, the prediction of adverse outcomes, in particular adverse outcomes related to the cardiovascular system are not predictable. Hence, there is a need for a diagnosis of such adverse outcomes, e.g. stroke, cardiac arrest, cardiac mortality and the like. Furthermore, the dialysis procedure is able to remove fluid from the body. Clinical assessment, e.g. of edema, alone is often not sufficient to determine the fluid status. Therefore, ultrasound of the V. cava inferior and respiration-dependent variability of the diameter is an indication of normovolemia. However, direct diagnosis of hypervolemia as well as other adverse outcomes is often cumbersome, time consuming and prone to subjective measures. The present invention now provides a tool that allows the diagnosis as well as the prognosis of such adverse outcomes due to the finding that midkine levels after cardiovascular stress have a prognostic and diagnostic value.

Midkine was discussed as biomarker for diverse diseases, especially within the context of cancer, cardiovascular and kidney diseases (Jones D R. Measuring midkine: the utility of midkine as a biomarker in cancer and other diseases; Br J Pharmacol. 2014; 171(12):2925-39). However, serum midkine levels in healthy subjects are mostly determined within a narrow range that depends on the applied detection system. Beyond these “background” levels, several diseases are known to be accompanied by elevated midkine serum levels. The main challenge to the use of midkine as diagnostic biomarker by single determinations is the generality of its regulation and lack of specificity for diseases. The present inventors have unexpectedly found that midkine levels are well suited as such biomarker if the subject underwent cardiovascular stress and that adverse outcomes specifically correlate with decreased levels of midkine after cardiovascular stress as compared to healthy controls, or patients undergoing dialysis with favorable outcome. Thus, the change in midkine levels during cardiovascular stress is indicative of the risk of acquiring adverse outcomes in the future.

### SUMMARY OF THE INVENTION

The inventors have found that midkine levels in samples of patients are indicative of adverse outcomes, e.g. of the cardiovascular system, like cardiovascular diseases or morbidity, if the samples have been taken after subjecting the subject to be diagnosed to cardiovascular stress. Hence, the present invention relates to a method for the prognosis of adverse outcomes in a subject comprising the following steps: (i) determining the midkine level in a sample of said subject; and (ii) comparing the determined midkine level to a control midkine level; wherein said sample is taken after the subject has been subjected to cardiovascular stress; wherein said control midkine level is derived from sample(s) from one or more subjects not showing adverse outcomes; and wherein a decreased determined midkine level as compared to the control midkine level is indicative of an adverse outcome.

The method may also combine the midkine levels before and after the cardiovascular stress. In this embodiment the difference of midkine levels is determined before and after the cardiovascular stress. The inventors found out that midkine levels increase during the cardiovascular stress. Hence, in one embodiment the method according to the invention determines the increase in midkine levels (also referred to as the Δmidkine value) in samples of subjects taken before and after the cardiovascular stress. In particular, the invention also relates to a method for the prognosis of adverse outcomes in a subject comprising the following steps: (i) determining the increase in midkine levels (Δmidkine value) in samples of said subject during cardiovascular stress; and (ii) comparing the determined Δmidkine value to a control Δmidkine value; wherein said control Δmidkine value is derived from one or more subjects not showing adverse outcomes; and wherein a decreased determined Δmidkine value as compared to the control Δmidkine value is indicative of an adverse outcome. The inventors found that an increase of midkine levels during the cardiovascular stress below average, e.g. below the 25^{th} percentile of a healthy population is indicative of an adverse outcome according to the invention, e.g. below a 10 fold increase. In one embodiment the invention relates to a method for the prognosis of adverse outcomes in a subject comprising the step of: determining the increase in midkine levels in samples of said subject during cardiovascular stress; wherein an increase in midkine levels in samples of said subject during cardiovascular stress below the 25^{th} percentile of a healthy populations is indicative of an adverse outcome, preferably an increase of less than 10 fold is indicative of an adverse outcome; more preferably of less than 8 fold, yet more preferably of less than 7 fold.

In one embodiment the cardiovascular stress is dialysis. In such embodiment the invention also relates to method for the prognosis of adverse outcomes in a subject undergoing dialysis therapy comprising the step of: determining the increase in midkine levels in samples of said subject during dialysis; wherein an increase in midkine levels in samples of said subject during dialysis of less than 10 fold is indicative of an adverse outcome; preferably of less than 8 fold, more preferably of less than 7 fold.

The present invention also relates to the use of a midkine antibody or an antigen binding fragment thereof for the prognosis of adverse outcomes in a subject undergoing dialysis therapy.

### FIGURE LEGENDS

FIG. 1. Serum midkine levels before and after dialysis treatment. All dialysis patients provided serum samples before and after two dialysis sessions following a short (2 day) and long (3 day) dialysis-free interval. (A) Individual changes of midkine serum levels are provided. Midkine serum levels after dialysis treatment increased following the short 2 day dialysis-free interval (p<0.001) and the long 3 day dialysis-free interval (p<0.001). (B) Correlation of midkine serum level changes during dialysis after the short and long dialysis-free interval (r²=0.33, p<0.001).

FIG. 2. Correlation of applied heparin with delta midkine levels. (A) The doses of applied non-fractionated heparin were correlated with changes in serum midkine levels after a short (r²=0.06, p=0.03) and long dialysis-free interval (r²=0.17, p<0.001). (B) For non-fractionated heparin similar correlations of delta midkine levels after a short (r²=0.002, p=0.88) and long dialysis-free interval (r²=0.01, p=0.76) were performed.

FIG. 3. Changes in serum midkine levels during dialysis in patients diagnosed with diabetes and/or hypervolemia. (A) For the diabetic (n=33) versus non-diabetic (n=50) patients subgroup analyses were performed. Absolute midkine levels were assessed after a short and long dialysis-free interval and Δmidkine values were calculated. Intergroup comparisons did not yield significant differences. (B) For the patients diagnosed with hypervolemia (n=51) versus euvolemia (n=32) subgroup analyses were performed. Absolute midkine levels were assessed after a short and long dialysis-free interval. Intergroup comparisons yielded significant differences after the short (p=0.05) and long interval (p=0.007). (C) For the patients diagnosed with diabetes and hypervolemia (n=19) versus those without diabetes and euvolemia (n=15) subgroup analyses were performed. Absolute midkine levels were assessed after a short and long dialysis-free interval. Intergroup comparisons yielded significant differences after the short (p=0.05) and long interval (p=0.001). (D) Δmidkine (midkine levels after dialysis-before dialysis) levels were calculated for the short and long dialysis-free interval. Here, significant differences for the subgroup comparisons were confirmed after the long interval (p<0.01).

FIG. 4. Kaplan-Meler survival curves. (A) Patients with less than average midkine levels after the long dialysis-free interval (group 1) were compared with those above average Δmidkine levels (group 2) over a 36 months observation period and censored for mortality (p=0.049 for intergroup comparison). (C) Patients with less than average Δmidkine levels after the long dialysis-free interval (group 1) were compared with those with above average Δmidkine levels (group 2) over a 36 months observation period and censored for cardiovascular mortality (p=0.03 for intergroup comparison). Patients belonging to the <25 percentile Δmidkine levels after the long dialysis-free interval (group 1) were compared with those with >75 percentile delta midkine levels (group 2) over a 36 months observation period and censored for overall mortality (B) and cardiovascular mortality (D). (E, F): overall and cardiovascular survival, respectively, for uPAR. (G, H): overall and cardiovascular survival, respectively, for NTproANP.

FIG. 5. Serum midkine levels before and after dialysis treatment. (A) Δmidkine values (midkine level post-pre-hemodialysis) were calculated for the short and long interval, yielding no significant difference. Furthermore the ΔΔmidkine levels (Δmidkine short interval-Δmidkine long interval) were calculated to assess the variability of midkine changes. (B) For the subgroups of diabetic (n=33) versus non-diabetic (n=50) patients analyses were performed. Δmidkine (midkine levels after dialysis-before dialysis) were calculated for the short and long dialysis-free interval. (C) For the subgroups of patients diagnosed with hypervolemia (n=51) versus euvolemia (n=32), Δmidkine (serum midkine levels after dialysis-before dialysis) were calculated for the short and long dialysis-free interval. Here, significant differences for the subgroup comparisons were confirmed.

FIG. 6. Kaplan-Meler survival curves. (A) Patients with less-than-average ADMA serum levels before the long dialysis-free interval (group 1) were compared with those having above-average ADMA levels (group 2). Over a 36 months observation period, censoring was performed for overall mortality and (B) cardiovascular mortality.

### DETAILED DESCRIPTION OF THE INVENTION

“Adverse outcomes” in context with the present invention relate to adverse outcomes, e.g. diabetes or an adverse outcome related to the cardiovascular systems, in particular to cardiovascular diseases. Preferably the term relates to cardiovascular diseases related to or conveying the risk of death. In a preferred embodiment the adverse outcome is selected from the group consisting of hypervolemia, cardiovascular mortality, mortality, myocardial infarction, stroke, and congestive heart failure; more preferably selected from the group consisting of hypervolemia, cardiovascular mortality and (overall) mortality. Cardiovascular mortality may be for example caused by myocardial infarction, stroke, congestive heart failure, sudden cardiac death, e.g. caused by arrhythmias, and the like.

“Hypervolemia” is well known by those skilled in the art. It relates to the fluid status of a subject. Hypervolemia subjects having a medical condition with too much fluid in the blood. The opposite condition is hypovolemia, which is too little fluid volume in the blood. Fluid volume excess in the intravascular compartment occurs due to an increase in total body sodium content and a consequent increase in extracellular (intravascular and interstitial) body water. The mechanism usually stems from compromised regulatory mechanisms for sodium handling as seen in congestive heart failure (CHF), kidney failure, and liver failure. It may also be caused by excessive intake of sodium from foods, intravenous (IV) solutions and blood transfusions, medications, or diagnostic contrast dyes. In the setting of patients with impaired kidney function, at advanced stages without replacement treatment or following initiation of renal replacement therapy, less than required fluid excretion by the kidneys may also convey fluid congestion. Treatment typically includes administration of diuretics and restriction of the intake of water, fluids, sodium, and salt. In the case of dialysis patients fluid removal by the dialysis procedure is the other means for correction of fluid disturbances, however only feasible when sufficient intravascular fluid is present and thus a sufficient high arterial blood pressure is present. The excess fluid, primarily salt and water, builds up in various locations in the body and leads to an increase in weight, swelling in the legs and arms (peripheral edema), and/or fluid in the abdomen (ascites). Eventually, the fluid enters the air spaces in the lungs (pulmonary edema) reduces the amount of oxygen that can enter the blood, and causes shortness of breath (dyspnea) or enters pleural space by transudation (pleural effusion which also causes dyspnea), which is the best indicator of estimating central venous pressure is increased. It can also cause swelling of the face. Fluid can also collect in the lungs when lying down at night, possibly making nighttime breathing and sleeping difficult (paroxysmal nocturnal dyspnea). In a preferred embodiment hypervolemia relates to a status where (i) the actual weight exceeds the clinically defined optimum (weight of the subject after dialysis) by >0.5 kg, and/or (ii) the vena cava diameter is more than 20 mm wide, and/or (iii) or a lung comet tail phenomenon is present (see Garbella E., et al.; Pulmonary edema in healthy subjects in extreme conditions; Pulm Med. 2011; 2011:275857. doi: 10.1155/2011/275857). It will be acknowledged by those of skills in the art that the prognosis or diagnosis of hypervolemia may also be employed in terms of a prognosis or diagnosis of related diseases or diseases known to trigger hypervolemia, e.g. those outlined above, like congestive heart failure, kidney failure or liver failure, such as heart failure, liver cirrhosis; or nephrotic syndrome.

“Prognosis” in context of the present invention relates to a prediction of an event in the future or the diagnosis of a certain state in a subject. For example, if the prognosis is supposed to be a prognosis of cardiovascular mortality, the skilled person will understand that the method relates to the prediction of this event in the future. However, for disease states such as hypervolemia, the methods of the present invention may also be used as a diagnosis, i.e. the determination of a current status. However, in a preferred embodiment “prognosis” relates to the prediction of an adverse outcome in the future, i.e. to the determination of the risk of a subject for suffering from the adverse outcome in the future (also referred to as risk stratification). In a further preferred embodiment within 36 months. The skilled person will acknowledge that the levels or values being indicative of an adverse outcome means that the subject has or has the risk of acquiring said adverse outcomes, i.e. the subject being prognosed for the adverse outcome.

The inventors found that the midkine levels are indicative for the prognosis of adverse outcomes according to the invention. Further the data presented show that decreased levels of midkine or Δmidkine value in samples taken after cardiovascular stress as compared to the respective control level or control Δmidkine value are indicative for adverse outcomes within 36 months from the date of the sampling. Hence, in a preferred embodiment the methods according to the present inventions are methods for prognosis of an adverse outcome within 36 months. This then also means that a decreased midkine level following cardiovascular stress is preferably indicative of an adverse outcome within 36 months. Preferably the methods according to the present inventions are methods for prognosis of a cardiovascular disease, preferably selected from the group of cardiovascular mortality within 36 months. This then also means that a decreased midkine level following cardiovascular stress is preferably indicative of an adverse outcome within 36 months.

The inventors have exemplified the invention by using dialysis as the cardiovascular stress. Dialysis is a model system for cardiovascular stresses. The dialysis removes liquid from the body, in particular from the cardiovascular system. The Examples herein exemplify the present invention using hemodialysis (also referred to as dialysis) as the cardiovascular stress. Dialysis is known to cause cardiovascular stress (Assa S, et al. Comparison of cardiac positron emission tomography perfusion defects during stress induced by hemodialysis versus adenosine. Am J Kidney Dis. 2012; 59(6):862-864). Due to the increased loss in body fluid the cardiovascular system is stressed and therefore causes in some cases adverse outcomes like cardiac arrest. Dialysis hence represents a suited model for cardiovascular stresses.

A “cardiovascular stress” refers to any stress subjected to the cardiovascular system of the subject. Such stress is preferably caused by loss of liquid and/or an increase in cardiac activity, e.g. measurable by increased pulse or increased blood pressure. The stress may be caused by different means, e.g. cardiovascular stimulating drugs (e.g. dobutamine), or by physical strain or physical stress (ergometry). Cardiovascular stimulating drugs are known by the skilled person and include sympathomimetic drugs, like β1-stimulating drugs (e.g. dobutamine, dipyridamol with atropin or adenosin). Physical strain or stress may be for example applied to the subject during cardiac stress testing, optionally accompanied by monitoring body functions, e.g. respiration, pulse, blood pressure etc. In a preferred embodiment the cardiovascular stress is selected from the group consisting of dialysis, hemodialysis, stress electrocardiogram, cardiac stress, and cardiac stress testing, administration of cardiovascular stimulating drugs, preferably adenosine, and dobutamine. In a further preferred embodiment the cardiovascular stress is hemodialysis.

In one embodiment the methods according to the present invention also comprise a step of subjecting the subject of interest to a cardiovascular stress according to the present invention, preferably before the sample for determining the level of midkine is taken, or between the time points at which at least two samples for determination of the Δmidkine value are taken.

“Subject” in the meaning of the invention is understood to be all persons or animals, whether or not they exhibit pathological changes, unless stated otherwise. For the purposes of the present invention the “subject” (or “patient”) may in particular be a vertebrate. In the context of the present invention, the term “subject” includes both humans and other animals, particularly mammals, and other organisms. Thus, the herein provided methods are applicable to both human therapy and veterinary applications. Accordingly, said subject may be an animal such as a horse, cat, dog, mouse, rat, hamster, rabbit, guinea pig, ferret, chicken, sheep, bovine species, camel, or primate. Preferably, the subject is a mammal. Most preferably the subject is human. In a further preferred embodiment of the invention the patient is a human suspected to have a risk to acquire or to suffer from an adverse outcome. As midkine levels are known to be altered in cancer patients, the subject is preferably cancer free, preferably the subject has not been diagnosed as having cancer within 2 years and is not under ongoing chemotherapy for treating cancer. In the meaning of the invention, any sample collected from cells, tissues, organs, organisms or the like can be a sample of a subject to be diagnosed (prognosed).

In a preferred embodiment further parameters may be assessed and included into the methods according to the present invention. A parameter is a characteristic, feature, or measurable factor that can help in defining a particular system. A parameter is an important element for health- and physiology-related assessments, such as a disease/disorder/clinical condition risk. Furthermore, a parameter is a characteristic that is objectively measured and evaluated as an indicator of normal body status or normal biological processes, pathogenic status or processes, or pharmacologic responses to a therapeutic intervention. An exemplary parameter can be selected from the group consisting of body mass index, weight, age, and/or sex.

In some aspects of the present invention, in addition to the midkine level, the levels of further (bio)markers are determined in the same sample. For example, the level(s) of urokinase-type plasminogen activator receptor (uPAR) (NCBI Reference Sequences of isoforms 1 to 4, respectively: NP_002650.1, NP_001005376.1, NP_001005377.1, NP_001287966.1) and/or pro-atrial natriuretic peptide (pro-ANP) (NCBI Reference Sequence NP_006163.1) or a fragment thereof such as N-terminal proatrial natriuretic peptide (NTproANP) can be determined in addition to the midkine level. In principle, for the determination of these further (bio)markers the same assay formats as for midkine can be used, particularly immunoassays and the same principles and considerations for the assessment of the prognosis of the adverse outcomes apply as for midkine and as outlined herein. In particular, an increased determined level of uPAR as compared to a control uPAR level is indicative of an adverse outcome. Similarly, an increased determined level of proANP or a fragment thereof such as NTproANP as compared to a control level of proANP or the fragment thereof such as NTproANP is indicative of an adverse outcome.

The combination of two or more markers may result in an improved prognosis.

It may be advantageous that subject has not been subjected to cardiovascular stress within a certain period preceding the actual cardiovascular stress before and/or after which the samples are to be taken according to the present invention. Hence, in a preferred embodiment the subject has not been subjected to a cardiovascular stress for at least 2 days prior the cardiovascular stress before and/or after the samples according to the present invention have been taken, preferably the methods of the invention are performed following a cardiovascular stress free interval of at least 2 days, preferably an interval of 2 days or of 3 days. In case of the method implicating the determination of a Δmidkine value, the determination is preferably started following a cardiovascular stress free interval of 2 days or of 3 days.

The skilled person will understand that the control levels are derived from samples which correspond to the samples of the subject to be diagnosed, e.g. if the sample of the subject to be diagnosed is taken after the cardiovascular stress, the samples from which the control levels are derived are also taken after a cardiovascular stress, preferably of the same kind. Hence, preferably the control midkine levels are derived from the sample(s) taken after said one or more subjects not showing adverse outcomes have been subjected to cardiovascular stress. In a further embodiment the samples the Δmidkine value have been taken before and after said one or more subjects not showing adverse outcomes have been subjected to cardiovascular stress.

In the context of the present invention, the levels of the (bio)markers, such as midkine, uPAR and proANP/NT-proANP, and in particular the control levels of the (bio)markers, such as midkine, uPAR and proANP/NT-proANP, or the control Δmidkine value may be analyzed in a number of fashions well known to a person skilled in the art. For example, each assay result obtained may be compared to a “normal” value, or a value indicating a particular disease or outcome. A particular diagnosis/prognosis may depend upon the comparison of each assay result to such a value, which may be referred to as a diagnostic or prognostic “threshold”. In certain embodiments, assays for one or more diagnostic or prognostic indicators are correlated to a prognosis or risk of adverse outcomes by merely the presence or absence of the indicator(s) in the assay. For example, an assay can be designed so that a positive signal only occurs above a particular threshold concentration of interest, and below which concentration the assay provides no signal above background. Presently, the assay may be designed that a signal is only given, if the level of midkine is below the control level, indicating for the risk or prognosis of adverse outcomes. Or, vice versa, a signal may be given only if the level of midkine is at or above the control level, the presence of the signal not indicating the risk or prognosis of adverse outcomes and the absence of the signal indicating the risk or prognosis of adverse outcomes.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy individuals not showing adverse outcomes, e.g. after cardiovascular stress) and “disease” populations (individuals showing adverse outcomes, e.g. after cardiovascular stress). For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, below which the test is considered to be abnormal and above which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art. See, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group.

The skilled artisan will understand that associating a diagnostic or prognostic indicator, with a diagnosis or with a prognostic risk of a future clinical outcome is a statistical analysis. For example, a marker level (e.g. the level of midkine or the Δmidkine value) of lower than X may signal that a patient is more likely to suffer from an adverse outcome than patients with a level more than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

The following discussions with respect to midkine levels equally apply to other (bio)markers such as uPAR and proANP/NT-proANP. However, in contrast to midkine, the levels of uPAR and proANP/NTproANP indicative for an adverse outcome are increased with respect to the control level.

Suitable threshold levels for the stratification of subjects into different groups (categories) have to be determined for each particular combination of midkine levels, disease and/or cardiovascular stress. This can e.g. be done by grouping a reference population of patients according to their level of midkine or Δmidkine value into certain quantiles, e.g. quartiles, quintiles or even according to suitable percentiles. For each of the quantiles or groups above and below certain percentiles, hazard ratios can be calculated comparing the risk for an adverse outcome, e.g. in terms of survival rate/mortality. In such a scenario, a hazard ratio (HR) above 1 indicates a higher risk for an adverse outcome for the patients. A HR below 1 indicates a low risk for an adverse outcome in the group of patients. A HR around 1 (e.g. +/−0.1) indicates no elevated risk for the particular group of patients. By comparison of the HR between certain quantiles of patients with each other and with the HR of the overall population of patients, it is possible to identify those quantiles of patients who have an elevated risk thereby attributing the subject for an adverse outcome according to the present invention.

In some cases presence adverse outcomes will affect patients with high levels or non-decreased levels of midkine or increased Δmidkine value after cardiovascular stress (e.g. above the 25^{th} percentile), while in other cases only patients with low levels or decreased levels of midkine or decreased Δmidkine value after cardiovascular stress will be affected (e.g. below the 25^{th} percentile). However, with the above explanations, a skilled person is able to identify those groups of patients having the risk of adverse outcomes and those who have not. Exemplarily, some combinations of some adverse outcomes and decreased midkine levels or Δmidkine values are disclosed in the appended examples. In another embodiment of the invention, the prognosis of an adverse outcome for a patient is determined by relating the patient's individual level of marker to certain percentiles (e.g. 2.5^{th} or 25% percentile) of a healthy population.

In a preferred embodiment of the present invention, midkine levels or Δmidkine values below the 25% percentile of levels or values of a population of subjects not having or showing adverse outcomes after cardiovascular stress (preferably within 36 months) are indicative of an adverse outcome according to the present invention. This is a particular preferred embodiment and the skilled person will acknowledge that it may be adapted to any embodiment of the methods according to the present invention.

Kaplan-Meier estimators may be used for the assessment or prediction of the outcome or risk (e.g. prognosis of adverse outcome) of a patient.

The inventors have found that in subjects in whom an adverse outcome according to the present invention is not to be expected the midkine levels are higher compared to the levels observed in samples of patients which showed adverse outcomes after the cardiovascular stress. The enclosed Examples show particular levels of midkine and Δmidkine values in subjects not showing adverse outcomes (after a cardiovascular stress) for examples of adverse outcomes and cardiovascular stress. In a preferred embodiment, the control levels according to the present invention are values as determined by the examples. The midkine control level, i.e. the level derived from sample(s) from one or more subjects not showing adverse outcomes, preferably said midkine control level is 33 ng/ml, preferably 30 ng/ml, more preferably 28 ng/ml.

Particular preferred combinations of the adverse outcomes and the midkine control level are exemplified in the annexed Examples. Even though the skilled person will acknowledge that these are not limiting, in a preferred embodiment the adverse outcome is (overall) mortality, and said midkine control level is 27 ng/ml, preferably 24 ng/ml even more preferably 20 ng/ml. Further preferred embodiments relate to the adverse outcome being cardiovascular mortality, and said midkine control level being 27 ng/ml, preferably 24 ng/ml, even more preferably 20 ng/ml, and particular preferred 18 ng/ml. It is further preferred that the cardiovascular stress is dialysis.

In one preferred embodiment the invention relates to a method for prognosis of adverse outcomes in a subject, comprising the step of determining the level of midkine in a sample of said subject; wherein the sample has been taken after subjecting the subject to a cardiovascular stress; wherein midkine levels of less than 33 ng/ml, ng/ml are attributed to the risk or prognosis of adverse outcomes in the subject, preferably midkine levels of 30 ng/ml or less, more preferably midkine levels of 28 ng/ml or less. It is further preferred that the cardiovascular stress is dialysis.

In a further preferred embodiment the invention relates to a method for prognosis of (overall) mortality in a subject, comprising the step of determining the level of midkine in a sample of said subject; wherein the sample has been taken after subjecting the subject to a cardiovascular stress; wherein midkine levels of less than 27 ng/ml, ng/ml are attributed to the risk or prognosis of (overall) mortality in the subject, preferably midkine levels of 24 ng/ml or less, more preferably midkine levels of 20 ng/ml or less. It is further preferred that the cardiovascular stress is dialysis.

In a further preferred embodiment the invention relates to a method for prognosis of cardiovascular mortality in a subject, comprising the step of determining the level of midkine in a sample of said subject; wherein the sample has been taken after subjecting the subject to a cardiovascular stress; wherein midkine levels of less than 27 ng/ml are attributed to the risk or prognosis of cardiovascular mortality in the subject, preferably midkine levels of 24 ng/ml or less, more preferably midkine levels of 20 ng/ml or less; particular preferred midkine levels of 18 ng/ml or less. It is further preferred that the cardiovascular stress is dialysis.

In a further preferred embodiment the invention relates to a method for prognosis of hypervolemia in a subject, comprising the step of determining the level of midkine in a sample of said subject; wherein the sample has been taken after subjecting the subject to a cardiovascular stress; wherein midkine levels of less than 32 ng/ml, ng/ml are attributed to the risk or prognosis of hypervolemia in the subject, preferably midkine levels of 28 ng/ml or less, more preferably midkine levels of 25 ng/ml or less. It is further preferred that the cardiovascular stress is dialysis.

Suitable cut-off values for uPAR above which the level is indicative for the respective adverse outcome are for example 1200 pg/ml, 1220 pg/ml, 1240 pg/ml, and 1260 pg/ml, preferably 1220 pg/ml.

Suitable cut-off values for NTproANP above which the level is indicative for the respective adverse outcome are for example 55 ng/ml, 59 ng/ml, 60 ng/ml, and 61 ng/ml, preferably 59 ng/ml.

The skilled person will understand that the levels determined may vary depending on the assay used. The levels and values herein represent preferred embodiments that are determined using a currently commercially available assay, preferably human midkine ELISA (ReproTech, Hamburg, Germany).

In one embodiment the presence of a cardiosvascular disease or diabetes in said subject to be diagnosed further indicates an adverse outcome.

In one embodiment the cardiovascular stress is dialysis (also referred to as hemodialysis). A method for the prognosis of adverse outcomes in a subject undergoing dialysis therapy comprising the step of determining the increase in midkine levels in samples of said subject during dialysis (Δmidkine value); wherein a Δmidkine value of less than 25 ng/ml, preferably of less than 20 ng/ml, more preferably of less than 10 ng/ml, is indicative of the prognosis for an adverse outcome.

In a preferred embodiment the determination of the Δmidkine value in the methods according to the invention comprises the following steps: (i) determining the level of midkine in a sample taken before the subject is subjected to a cardiovascular stress (midkine level 1): (i) determining the level of midkine in a sample taken after the subject has been subjected to a cardiovascular stress (midkine level 2); and (iii) calculating the difference between the two determined midkine levels and thereby obtaining the Δmidkine value. As outlined herein, cardiovascular stress causes an increase in midkine levels. This means that midkine level 2 is expected to be higher or increased as compared to midkine level 1.

“equal” in context with the present invention means that the midkine levels or the Δmidkine value differ by not more than ±10%, preferably by not more than ±5%, more preferably by not more than ±2%. “Decreased” or “increased” in the context of the present invention mean that the midkine levels or the Δmidkine value differ by more than 10%, preferably by more than 15%, preferably more than 20%.

It will be readily understood by the skilled person that the control levels from subjects having the desired disease or response and to which the determined levels are compared to, are not necessarily determined in parallel but may be represented by previously determined levels. Nevertheless, control levels may be determined in parallel. The skilled person with the disclosure of the present invention and his knowledge is able to determine such levels, as will be outlined herein below. Hence, the control levels of the present invention may be previously defined thresholds. Preferred thresholds are disclosed herein.

The “sample” according to the invention is preferably selected from the group consisting of blood sample, serum sample, and plasma sample, urine sample; most preferably serum sample.

Where appropriate, the sample may need to be homogenized, or extracted with a solvent prior to use in the present invention in order to obtain a liquid sample. A liquid sample hereby may be a solution or suspension. Liquid samples may be subjected to one or more pre-treatments prior to use in the present invention. Such pre-treatments include, but are not limited to dilution, filtration, centrifugation, concentration, sedimentation, precipitation, dialysis. Pre-treatments may also include the addition of chemical or biochemical substances to the solution, such as acids, bases, buffers, salts, solvents, reactive dyes, detergents, emulsifiers, and/or chelators.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood) for at least 15 minutes at 2000 to 3000 g.

“Serum” is the liquid fraction of whole blood that is collected after the blood is allowed to clot. When coagulated blood (clotted blood) is centrifuged serum can be obtained as supernatant. It does not contain fibrinogen, although some clotting factors remain.

The skilled person knows that it might be desirable to stabilize the sample after taking, i.e. in order to prevent the analyte(s) from degeneration. This might be achieved through direct preparation and analysis and/or the addition of stabilizing agents. “Stabilizing” in context with the present invention in particular refers to stabilization of proteins, in particular midkine. Means and methods to stabilize a sample such as a whole blood, plasma or serum sample are known by the skilled person. It may also be preferred that the serum or plasma is prepared from the whole blood sample directly (i.e. within 30 min or less, e.g. 5 min or less) after it has been taken and then either analyzed directly or stabilized. Stabilizing may occur through addition of stabilizing agents or by storage under stabilizing conditions, e.g. at low temperatures, such as 4° C. or less, −20° C. or less, or −80° C. or less. In one particular preferred embodiment the sample is a serum sample directly prepared after taking the respective whole blood sample. Further preferred, the serum sample is analyzed immediately after preparation or stored after preparation until analysis at −20° C. or less, more preferably at −80° C. or less. As freeze-thaw cycles may also influence sample and analyte stability, it is further preferred that the samples are only thawn once prior analysis.

It will be readily understood by the skilled person that the control levels according to the present invention are preferably derived from the same type of sample as the sample used in the method of the invention; e.g. if the sample taken from the subject to be diagnosed is a serum sample than the control level is preferably also derived from one or more serum samples. Furthermore, the absolute levels or values referred to herein are based on the findings in the Examples of the present application. The therein used sample types were serum samples. Hence, the absolute midkine levels (ng/ml) or Δmidkine value (ng/ml) are preferably levels or values in or of serum samples.

Midkine (MK or MDK) also known as neurite growth-promoting factor 2 (NEGF2) is a protein that in humans is encoded by the MDK gene (Kaname T, et al. (August 1993). “Midkine gene (MDK), a gene for prenatal differentiation and neuroregulation, maps to band 11p11.2 by fluorescence in situ hybridization”. Genomics 17 (2): 514-515). Midkine is a basic heparin-binding growth factor of low molecular weight, and forms a family with pleiotrophin (NEGF1, 46% homologous with MK). It is a nonglycosylated protein, composed of two domains held by disulfide bridges. It is a developmentally important retinoic acid-responsive gene product strongly induced during mid-gestation, hence the name midkine. Restricted mainly to certain tissues in the normal adult, it is strongly induced during oncogenesis, inflammation and tissue repair.

MK is pleiotropic, capable of exerting activities such as cell proliferation, cell migration, angiogenesis and fibrinolysis. A molecular complex containing receptor-type tyrosine phosphatase zeta (PTPζ), low density lipoprotein receptor-related protein (LRP1), anaplastic leukemia kinase (ALK) and syndecans is considered to be its receptor (Muramatsu Takashi (2002). “Midkine and pleiotrophin: two related proteins involved in development, survival, inflammation and tumorigenesis”. J. Biochem. (Tokyo) 132 (3): 359-71). MK is involved in cancer and appears to enhance the angiogenic and proliferative activities of cancer cells (Kato M, Maeta H, Kato S, Shinozawa T, Terada T (October 2000). “Immunohistochemical and in situ hybridization analyses of midkine expression in thyroid papillary carcinoma”. Mod. Pathol. 13 (10): 1060-5). The expression of MK (mRNA and protein expression) has been found to be elevated in multiple cancer types, such as neuroblastoma, glioblastoma, Wilms' tumors, thyroid papillary carcinomas, colorectal, liver, ovary, bladder, breast, lung, esophageal, stomach, and prostate cancers. Serum MK in normal individuals is usually less than 0.5-0.6 ng/ml, whereas patients with these malignancies have much higher levels than this. In some cases, these elevated levels of MK also indicate a poorer prognosis of the disease, such as in neuroblastoma, gliablastoma, and bladder carcinomas. In neuroblastoma, for example, the levels of MK are elevated about three times the level in Stage 4 of the cancer (one of the final stages) than they are in Stage 1 (Ikematsu S, et al. (2003). “Correlation of elevated level of blood midkine with poor prognostic factors of human neuroblastomas”. Br. J. Cancer 88 (10): 1522-6). MK is a secreted protein has bee proposed as a target for cancer treatment as a result of its cancerous proliferation properties (Ireson C R, Kelland L R (2006). “Discovery and development of anticancer aptamers”. Mol. Cancer Ther. 5 (12): 2957-2962). Midkine as used herein refers to any midkine variant or allele, preferably to any human midkine variant or allele, in a preferred embodiment has the sequence of SEQ ID NO:1 as disclosed herein below.

Levels of midkine in samples such as may be detected by any method suitable for specifically detecting the presence or level of a protein. Such methods are known by the person skilled in the immunoassays come in many different formats and variations. An immunoassay for midkine detection and determination of its levels is commercially available (PeproTech, Hamburg; Catalog Number: 900-K190) may be preferred in the present invention.

“Immunoassays” in the meaning of the invention are assays utilizing the specific interaction between the midkine protein or antigenic fragments thereof and an antibody specifically binding said midkine protein or antigenic fragments thereof (anti-midkine antibody), in order to detect the presence or determine the concentration of midkine. For example, the detection and quantification of midkine can be performed with the aid of said anti-midkine antibodies or antigen binding fragments thereof, e.g. by immunoprecipitation or immunoblotting. For example, immunoassays in the meaning of the invention can be subdivided into the following steps: (1) midkine/anti-midkine antibody reaction, (2) if required separation of the midkine/anti-midkine antibody complex from other components of the reaction mixture especially from non-bound anti-midkine antibody and midkine and (3) measuring the response. As for the midkine/anti-midkine antibody reaction various configurations of passable, e.g. (a) precipitation of one reaction with an access of the other or (b) competition between known quantities of midkine or anti-midkine antibody and the material to be investigated. Immunoassays are known in the art and refer to an assay in which a certain analyte is detected using specific antibody antigen interaction, i.e. the binding of an antibody to its antigen. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays. art and include immunoassays. In a preferred embodiment the levels of midkine are detected in an immunoassay.

In the context of the immunoassays of the present invention the “anti-midkine antibody” may be present in its natural cellular environment and can be used together with the material associated with antibodies in its natural state as well as in isolated form with respect to its primary, secondary and tertiary structures. The anti-midkine antibodies are well known to those skilled in the art. The antibody is preferably used in isolated form, i.e. essentially free of other proteins, lipids, carbohydrates or other substances naturally associated with anti-midkine antibodies. “Essentially free of” means that the receptor is at least 75%, preferably at least 85%, more preferably at least 95% and especially preferably at least 99% free of other proteins, lipids, carbohydrates or other substances naturally associated with the anti-midkine antibody.

The term “antibody” comprises monoclonal and polyclonal antibodies and binding fragments thereof, in particular Fc-fragments as well as so called “single-chain-antibodies” (Bird R. E. et al (1988) Science 242:423-6), chimeric, humanized, in particular CDR-grafted antibodies, and dia or tetrabodies (Holliger P. et al (1993) Proc. Natl. Acad. Sci. U.S.A. 90:6444-8). Also comprised are immunoglobulin like proteins that are selected through techniques including, for example, phage display to specifically bind to the polypeptides of the present invention. In this context the term “specific binding” refers to antibodies raised against peptides derived from midkine. Such peptides can comprise additional or less N- or C-terminal amino acids. An antibody is considered to be specific to midkine, if its affinity towards the variant it is at least 50-fold higher, preferably 100-fold higher, more preferably at least 1000-fold higher than towards the full length human or murine midkine, preferably human midkine. Preferably specific antibodies of the present invention do not or essentially do not bind to full length human midkine; preferably to a polypeptide comprising the sequence of SEQ ID NO:1 or comprising a sequence showing at least 80% sequence identity thereto, preferably at least 90%, more preferably at least 95%, yet more preferably at least 97%, even more preferred at least 99% sequence identity to SEQ ID NO: 1. It is well known in the art how to make antibodies and to select antibodies with a given specificity. The anti-midkine antibody specifically binds to midkine or an antigenic fragment thereof. Binding occurs through binding of the epitope on the protein or fragment by the antibody or at least the epitope binding fragment thereof. The antibody can also be modified (e.g. oligomeric, reduced, oxidized and labeled antibodies). The term anti-midkine antibody as used herein comprises both intact molecules and also anti-midkine antibody fragments such as Fab, F(ab′)₂ and Fv capable of binding specific epitope determinants of the midkine. In these fragments the anti-midkine antibody(ies) capability of selectively binding its antigen or receptor is retained in part, the fragments being defined as follows: (1) Fab, the fragment which contains a monovalent antigen-binding fragment of an antibody molecule, can be generated by cleavage of a whole antibody using the enzyme papaine, thereby obtaining an intact light chain and part of a heavy chain; (2) the Fab fragment of an antibody molecule can be produced by treatment of a whole antibody with pepsin and subsequent reduction, thereby obtaining an intact light chain and part of a heavy chain, two Fab fragments per antibody molecule are obtained; (3) F(ab′)₂ the fragment of the antibody which can be obtained by treatment of a whole antibody with the enzyme pepsin without subsequent reduction, F(ab′)₂ is a dimer comprised of two Fab fragments held together by two disulfate bonds; (4) Fv defined as fragment modified by genetic engineering which includes the variable region of the light chain and the variable region of the heavy chain is expressed in the form of two chains; and (5) single-chain antibody (SCA) defined as a molecule modified by genetic engineering, which includes the variable region of the light chain and the variable region of the heavy chain, linked by a suitable polypeptide linker to perform a genetically fused single-chain molecule. Also included in the term anti-midkine antibody(ies) are diabodies, single-domain antibody(ies) (sdAb, also referred to as Nanobody(ies)).

The term “epitope” as used in the present invention represents any antigen determinant on the Midkine. Epitope determinance normally consists of chemically active surface groups of molecules such as amino acids or sugar-side chains and normally has specific features of the free dimensional structure as well as specific chart properties.

The anti-midkine antibody binds specifically to the midkine or in doing so shows specific immuno reactivity when the anti-midkine antibody assumes its function in a binding reaction in the presence of a heterogeneous population of midkines or fragments thereof, thereby allowing a conclusion whether the midkine or another biological structure is present. Under the present conditions of an immunoassay, the above-mentioned anti-midkine antibodies will preferably bind to a specific portion of the midkine, while no significant binding to other proteins present in the sample will take place.

The determination of percent identity between two sequences is accomplished using the mathematical algorithm of Karlin and Altschul (1993) Proc. Natl. Acad. Sci. USA 90: 5873-5877. Such an algorithm is incorporated into the BLASTN and BLASTP programs of Altschul et al. (1990) J. Mol. Biol. 215: 403-410. BLAST nucleotide searches are performed with the BLASTN program, score=100, word length=12, to obtain nucleotide sequences homologous to the EPO variant polypeptide encoding nucleic acids. BLAST protein searches are performed with the BLASTP program, score=50, wordlength=3, to obtain amino acid sequences homologous to the EPO variant polypeptide, respectively. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used.

The term “peptide” or “polypeptide” of midkine used in the present invention, comprises also molecules differing from the original sequence by deletion(s), insertion(s), substitution(s) and/or other modifications well known in the prior art and/or comprising a fragment of the original amino acid molecule, the midkine still exhibiting the properties mentioned above, preferable binding to anti-midkine antibodies and being thereby detectable. Such a peptide has preferably at least a length of 100 amino acid residues but may also be shorter, e.g. at least 12, 15, 20 or 25 amino acid residues in length or even longer. Also included are allele variants and modifications. In the present specification all of the above illustrated modifications of the midkine will be referred to as “functionally analogous peptides or proteins” in brief.

The antibodies to detect and determine the levels of midkine according to the present invention are directed against midkine. This means that the antibodies specifically bind midkine. Specific binding of an antibody normally occurs via binding of a binding site of the antigen. The antibodies of the present invention are those specifically binding to midkine or immunogenic (antigenic) fragments thereof. This binding may occur via recognition of sequence or structural epitopes. The skilled person is aware of methods of how to determine specific epitopes, e.g. fragments of the antigen midkine, which are recognized and bound by the antibodies to be determined. Fragments of midkine binding to the antibodies are called immunogenic or antigenic fragments. Methods for determining fragments of an antigen binding the antibody are described in several publications (e.g. Gershoni, J M; Roitburd-Berman, A; Siman-Tov, DD; Tarnovitski Freund, N; Weiss, Y (2007). “Epitope mapping: The first step in developing epitope-based vaccines”. BioDrugs 21 (3): 145-56; Westwood, MR; Hay, FC (2001). Epitope Mapping: a practical approach. Oxford, Oxfordshire: Oxford University Press. ISBN 0-19-963652-4; Flanagan et al. (2011), “Mapping Epitopes with H/D-Ex Mass Spec”. Genetic Engineering and Biotechnology news; 31(1); Gaseitsiwe, S.; Valentini, D.; Mahdavifar, S.; Reilly, M.; Ehrnst, A.; Maeurer, M. (2009) “Peptide Microarray-Based Identification of Mycobacterium tuberculosis Epitope Binding to HLA-DRB1*0101, DRB1*1501, and DRB1*0401”. Clinical and Vaccine Immunology 17 (1): 168-75; Linnebacher, Michael; Lorenz, Peter, Koy, Cornelia; Jahnke, Annika; Born, Nadine; Steinbeck, Felix; Wollbold, Johannes; Latzkow, Tobias et al. (2012). “Clonality characterization of natural epitope-specific antibodies against the tumor-related antigen topoisomerase IIa by peptide chip and proteome analysis: A pilot study with colorectal carcinoma patient samples” Analytical and Bioanalytical Chemistry 403 (1): 227-38; Cragg, M. S. (2011). “CD20 antibodies: Doing the time warp”. Blood 118 (2): 219-20; Banik, Soma S. R.; Doranz, Benjamin J. (2010). “Mapping Complex Antibody Epitopes”. Genetic Engineering and Biotechnology News 3 (2): 25-8; and Paes, Cheryl; Ingalls, Jada; Kampani, Karan; Sulli, Chidananda; Kakkar, Esha; Murray, Meredith; Kotelnikov, Valery; Greene, Tiffani A. et al. (2009). “Atomic-Level Mapping of Antibody Epitopes on a GPCR”. Journal of the American Chemical Society 131 (20): 6952-4). In context with the present invention anti-midkine antibodies are understood as any immunoglobulin specifically recognizing/binding to midkine. The antibody in a preferred embodiment binds any midkine variant or allelic variant, preferably to SEQ ID NO: 1.

The skilled person will understand that controls for comparing the determined levels may be of different nature e.g. depending on the assay used. The kit according to the present invention may for example comprise one or more controls comprising midkine at the desired control level. Furthermore, the kit may comprise one or more standard solutions each solution comprising midkine at different levels, such standard solutions are particularly preferred in cases were a standard curves are to be applied. Exemplary dilutions and levels for such standard solutions are outlined herein above.

The embodiments set out for the immunoassays apply also to the kit of the invention. The kits of the present invention are meant for the detection of midkine in samples of a subject, preferably blood, serum, or plasma. Hence, in one embodiment they comprise means for the preparation of blood, e.g. for gaining serum or plasma thereof. Furthermore, the kit may comprise control composition and/or standards. The control composition preferably comprises midkine as positive control. Furthermore, the kit may comprise one or a plurality of standard compositions.

Such kits can comprise a carrier, package or container that is compartmentalized to receive one or more containers such as vials, tubes, and the like, each of the container(s) comprising one of the separate elements to be used in the method. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for either in vivo or in vitro use, such as those described herein. Directions and or other information can also be included on an insert which is included with the kit.

In a preferred embodiment the kit comprises an anti-midkine antibody or a functional analog thereof, optionally immobilized on a surface, preferably as defined above and specifically binding to midkine present in samples of subjects to be diagnosed, in particular present at increased levels of subjects which were subjected to a cardiovascular stress.

The immunological test kit according to the invention comprises the anti-midkine antibodies or a functional analog thereof or peptides or proteins of analogous function per se. The test kit of the invention comprises at least one anti-midkine antibody as defined above, optionally bound to a solid phase. Furthermore, the test kit may also comprise buffers, specific conjugate together with an enzyme, wash solution, substrate solution to detect the immune reaction and/or a quenching solution. Using these substances a person skilled in the art will be able to perform, e.g. an ELISA to detect the midkine and determine the level. The buffers, specific conjugate plus enzyme, wash solution, substrate solution to detect immune reaction and quenching solution are well known to those skilled in the art. For example, it would be sufficient to have the test comprise a freeze-dried anti-midkine antibody and to add the buffers and other solutions immediately prior to testing the biological material. However, it is also possible to provide the test kit with the anti-midkine antibody or its functionally analogous peptides of proteins bound to a solid phase. To detect the midkine the specific conjugate, wash solution, substrate solution and quenching solution, which can be components of the test kit, have to be added according to a mode well known to those skilled in the art.

In another advantageous embodiment of the invention, it is envisioned that the test kit is a test strip comprising the anti-midkine antibody or its functionally analogous peptides or proteins immobilized on a solid phase. For example, the test strip can be immersed in serum or other patient samples and incubated. Using a specific biochemical reaction on the test strip after formation of the midkine/anti-midkine antibody complex, a specific color reaction can be triggered by means of which the anti-midkine antibody can be detected and optionally the levels can be determined. The determination of the level may be accomplished through contruction of the strip in a way that the color reaction is only triggered at levels above or below a certain control level as further defined herein. The test system of the invention permits quantification of the midkine directly in a sample, e.g. in plasma of patients. The detection method according to the invention is time saving and cost effected. Large amounts of the samples can be tested and, owing to the low amount of the equipment required, routine laboratories can be used.

In a preferred embodiment of the invention the immunoassay is selected from the group of immunoprecipitation, enzyme immunoassay (EIA), radioimmunoassay (RIA), enzyme-linked immunosorbent assay (ELISA), fluorescent immunoassay, a chemiluminescent assay, an agglutination assay, nephelometric assay, turbidimetric assay, a Western Blot, a competitive immunoassay, a noncompetitive immunoassay, a homogeneous immunoassay a heterogeneous immunoassay, a bioassay and a reporter assay such as a luciferase assay or luminex.

General processes to detect an analyte or determine its level in a sample are known by the skilled person. Preferably they comprise a step in which an agent specifically detecting the analyte is contacted with the sample which is thought to comprise the analyte. Thereafter it is to be detected whether the agent has bound the analyte and hence forms a complex. Hence, in a preferred embodiment of the invention the methods according to the invention comprise the steps of (a) contacting the sample with a midkine antibody or an antigen binding fragment thereof under conditions allowing for the formation of a complex between said midkine antibody or the antigen binding fragment thereof with the midkine; and (b) detecting the formed complex. Preferably these steps relate to the determination of midkine levels in the sample as comprised in the methods according to the invention.

In a preferred embodiment the midkine antibody or the antigen binding fragment thereof is immobilized on a surface.

It is further preferred that the complex is detected using a secondary antibody against midkine. The skilled artisan will acknowledge that it might be preferred that the secondary antibody binds a different epitope of midkine than the “midkine antibody”, which is optionally immobilized. The secondary antibody is preferably labeled with a detectable marker.

The immunoassays can be homogenous or heterogeneous assays, competitive and non-competitive assays. In a particularly preferred embodiment, the assay is in the form of a sandwich assay, which is a non-competitive immunoassay, wherein the midkine protein (i.e. the “analyte”) to be detected and/or quantified is allowed to bind to an immobilized midkine antibody or antigen binding fragment thereof and to a secondary antibody or antigen binding fragment thereof. The midkine antibody or antigen binding fragment thereof (i.e. antibody), may e.g., be bound to a solid phase, e.g. a bead, a surface of a well or other container, a chip or a strip, and the secondary antibody is an antibody which is labeled, e.g. with a dye, with a radioisotope, or a reactive or catalytically active moiety such as a peroxidase, e.g. horseradish peroxidase. The amount of labeled antibody bound to the analyte is then measured by an appropriate method. The general composition and procedures involved with “sandwich assays” are well-established and known to the skilled person (The Immunoassay Handbook, Ed David Wild Elsevier LTD, Oxford; 3rd ed. (May 2005), ISBN-13: 978-0080445267; Hultschig C et al., Curr Opin Chem Biol. 2006 February; 10(1):4-10. PMID: 16376134, incorporated herein by reference). Sandwich immunoassays can for example be designed as one-step assays or as two-step assays.

The “sensitivity” of an immunoassay relates to the proportion of actual positives which are correctly identified as such, i.e. the ability to identify positive results (true positives positive results/number of positives). Hence, the lower the concentrations of the analyte that can be detected with an assay, the more sensitive the immunoassay is. The “specificity” of an immunoassay relates to the proportion of negatives which are correctly identified as such, i.e. the ability to identify negative results (true negatives/negative results). For an antibody the “specificity” is defined as the ability of an individual antigen binding site to react with only one antigenic epitope. The binding behaviour of an antibody can also be characterized in terms of its “affinity” and its “avidity”. The “affinity” of an antibody is a measure for the strength of the reaction between a single antigenic epitope and a single antigen binding site. The “avidity” of an antibody is a measure for the overall strength of binding between an antigen with many epitopes and multivalent antibodies.

The detectable label may for example be based on fluorescence or chemiluminescence. The labelling system comprises rare earth cryptates or rare earth chelates in combination with a fluorescence dye or chemiluminescence dye, in particular a dye of the cyanine type. In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in Kirk-Othmer, Encyclopedia of chemical technology, 4^{th} ed., executive editor. J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters.

It was an object of the invention to provide an in vitro method, in vitro diagnosis, prognosis, risk assessment, risk stratification, therapy control and/or operative control of a disorder or medical condition in a subject and/or a patient, which provides reliable information especially to the medical practitioner in the Emergency Department (ED) or Intensive Care Unit (ICU). Thus, the invention relates to the method for in vitro diagnosis, prognosis, risk assessment, risk stratification, therapy control and/or operative control of a disorder or medical condition in a subject, wherein the risk for an adverse outcome is determined by the herein provided method.

In certain aspects, the invention relates to the herein provided method to predict the mortality risk of a subject, wherein the prognosis of an adverse outcome of said subject is determined by the herein provided method. In certain aspects, the invention relates to a method used as a warning system for physician and clinicians to take appropriate therapy actions immediately, wherein said prognosis of said subject is determined by the herein provided method.

The invention also relates to the use of a midkine antibody or an antigen binding fragment thereof or a kit comprising either for the prognosis of adverse outcomes in a subject undergoing dialysis therapy. Preferably the use relates to the use in a method according to the present invention.

In certain aspects, the invention relates to the herein provided method for the use in treatment or prevention of disorder or a medical condition that is selected from the group consisting of adverse outcomes as defined herein above. The terms “treatment”, “therapy”, “prevention” and the like are used herein to generally mean obtaining a desired pharmacological and/or physiological effect. The effect may be prophylactic in terms of completely or partially preventing a disease/medical condition/disorder or symptom thereof and/or may be therapeutic in terms of partially or completely curing a disease/medical condition/disorder and/or adverse effect attributed to the disease/medical condition/disorder. The term “treatment” as used herein covers any treatment of a disease/medical condition/disorder in a subject and includes: (a) preventing and/or ameliorating the disease/medical condition/disorder in a subject which may be predisposed to the disease/medical condition/disorder, (b) inhibiting the disease/medical condition/disorder, i.e. arresting its development; or (c) relieving the disease/medical condition/disorder, i.e. causing regression of the disease/medical condition/disorder. The inventors found a link between reduced midkine levels after cardiovascular stress and adverse outcomes, e.g. cardiovascular mortality or hypervolemia. Hence, it is instantly plausible that the administration of midkine provides for a therapeutic benefit in prevention and treatment of adverse outcomes. Hence, in one embodiment the invention relates to midkine for use in the treatment or the prevention of an adverse outcome, wherein midkine is administered to the subject, if the subject exhibits midkine levels in a sample taken after the subject being subjected to a cardiovascular stress indicative of an adverse outcome or if the subject exhibits an increase of midkine levels during cardiovascular stress (Δmidkine values) being indicative of an adverse outcome. Preferably, the invention relates to midkine for use in the treatment or the prevention of an adverse outcome, wherein the subject has been prognosed for an adverse outcome in a method according to the present invention as outlined above. The midkine is preferably administered during or after cardiovascular stress. Furthermore, the invention also relates to a method of treatment or prevention of an adverse outcome, comprising the step of administering to a subject midkine; wherein midkine is administered to the subject, if the subject exhibits midkine levels or Δmidkine values in samples being indicative of an adverse outcome, the samples being taken after the subject being subjected to a cardiovascular stress being indicative of an adverse outcome. Preferably the midkine being administered if the subject has been prognosed for an adverse outcome in a method according to the present invention as outlined above. The midkine to be administered may be as defined further above, optionally as a pharmaceutically acceptable salt thereof. Furthermore, the administrated formulation may comprise pharmaceutically acceptable additives or adjutants, e.g. for formulation stabilization.

The invention is further illustrated by the included, non-limiting Examples and Figures. Furthermore, any reference cited herein is incorporated by reference into the present disclosure.

Disclosed Sequences

| SEQ ID NO: 1 | |
|---|---|
| 1 | MQHRGFLLLT LLALLALTSA VAKKKDKVKK GGPGSECAEW AWGPCTPSSK |
| 51 | DCGVGFREGT CGAQTQRIRC RVPCNWKKEF GADCKYKFEN WGACDGGTGT |
| 101 | KVRQGTLKKA RYNAQCQETI RVTKPCTPKT KAKAKAKKGK GKD |

### Examples

The present study was set up to test for inter- and intra-individual changes in midkine levels in subjects undergoing cardiovascular stress. To this end serum samples were collected immediately before and after the cardiovascular stress (exemplified with dialysis). A variation in volume status was anticipated as measurements were performed following short (2 day) and long (3 day) dialysis-free intervals. The midkine release would therefore be correlated with high cardiovascular stress by fluid removal during cardiovascular stress. Correlative analyses included clinical parameter on fluid status (comet tail phenomenon of lungs, V. cava collapse, clinical assessment), co-morbidities and follow-up mortality over 36 months.

Methods

Study Design

The study was approved by the local ethics committee (EK 73/90). 83 patients who underwent chronic hemodialysis thrice weekly at the KfH Magdeburg were enrolled following informed written consent. Clinical data were retrieved from the medical records. Fluid status was assessed by the caring physician and judged as overloaded when the optimal weight was exceeded by >0.5 kg. Ultrasound of the abdomen and quantification of the V. cava width was performed with >20 mm indicating fluid overload. A thoracic ultrasound was performed to demonstrate the comet tail phenomena, i.e. hyperechogenicity reflecting pulmonary edema. A carotid intima-media thickness >0.9 mm was defined as increased. Cancer was defined as being current when diagnosis occurred within <2 years and/or chemotherapy was ongoing. Diabetes mellitus was diagnosed according to the German Diabetes Society guidelines (Sacks D B, et al. Guidelines and recommendations for laboratory analysis in the diagnosis and management of diabetes mellitus. Diabetes Care. 2011; 34(6):e61-99). Reference levels for C-reactive protein (CRP) were below 0.5 mg/dl. Following informed written consent patients from a second cohort (n=88) were included in the study following a dialysis free 3 day interval and clinically scored as eu- or hypervolemic.

Analytical Methods

Blood was collected before and after dialysis, serum immediately prepared by centrifugation and samples stored at −80° C. Healthy donor serum (n=100) was collected for comparison.

Serum midkine levels were quantified using a human midkine ELISA (PeproTech, Hamburg, Germany) and measured on a TECAN Infinite 200 spectrophotometer (detection limit: 0.1 to 2,000 ng/ml). Blood counts were analyzed using a Coulter Counter (Beckman Coulter).

Serum urokinase-type plasminogen activator receptor (uPAR) and N-terminal proatrial natriuretic peptide (NTproANP) levels were quantified simultaneously in a multiplex format using a high sensitivity cytokine/chemokine kit (Magnetic Luminex Screening Assay, human premixed multi analyte kit, R&D Systems) according to the manufacturer's instructions (BioPlex200 Analyzer, BioRad). Values for analytes were determined from a standard curve of log dose versus median fluorescent intensity using a 5 parameter logistic fit.

Plasma concentrations of (a)symmetrical dimethylarginine (ADMA, SDMA), and L-arginine were measured as described (Martens-Lobenhoffer J, and Bode-Boger S M. Fast and efficient determination of arginine, symmetric dimethylarginine, and asymmetric dimethylarginine in biological fluids by hydrophilic-interaction liquid chromatography-electrospray tandem mass spectrometry. Clin Chem. 2006; 52(3):488-93).

Statistical Analysis

Data are presented as proportions for categorical variables and means±SD or medians (interquartile range, 25^{th}-75^{th} percentile) for continuous variables. For comparison of linear variables, the t-test or Mann-Whitney U-test were applied. Correlation analyses were conducted using Pearson (r) or Spearman (ρ) tests. The data were transformed logarithmically to achieve normal distributions, whenever possible. A p-value <0.05 was considered significant. Furthermore, for estimating the optimal cut-off point of uPAR, ΔMidkine and NTproANP to predict survival or normvolumina/hypervolumina, receiver operating characteristic (ROC) curves were calculated to quantify AUC values. Binary logistic regression analysis was used to identify associations.

Results

The bibliographic data for 83 patients are summarized in Table 1. The mean age was 64 years (±16), 65% were males. Participants underwent regular dialysis for 4.1 years on average [range: 0.2-22 years]. Anticoagulants applied for hemodialysis were non-fractionated (70 patients) and fractionated heparin (12 patients), or citrate (1 patient). Co-morbidities retrieved from medical records were arterial hypertension (98%), diabetes mellitus (40%), coronary artery disease (48%), and cerebrovascular disease (21%). 37/83 (45%) received ACE inhibitors or angiotensin type 1 receptor blockers (ARBs).

Midkine Serum Levels are Elevated in Dialysis Patients and Further Rise During Dialysis.

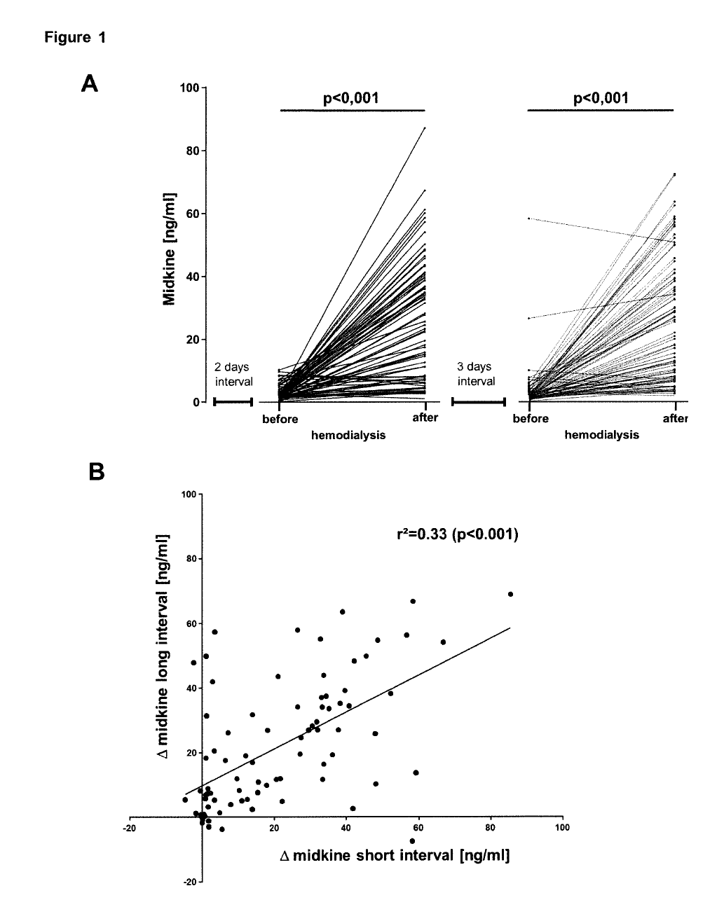

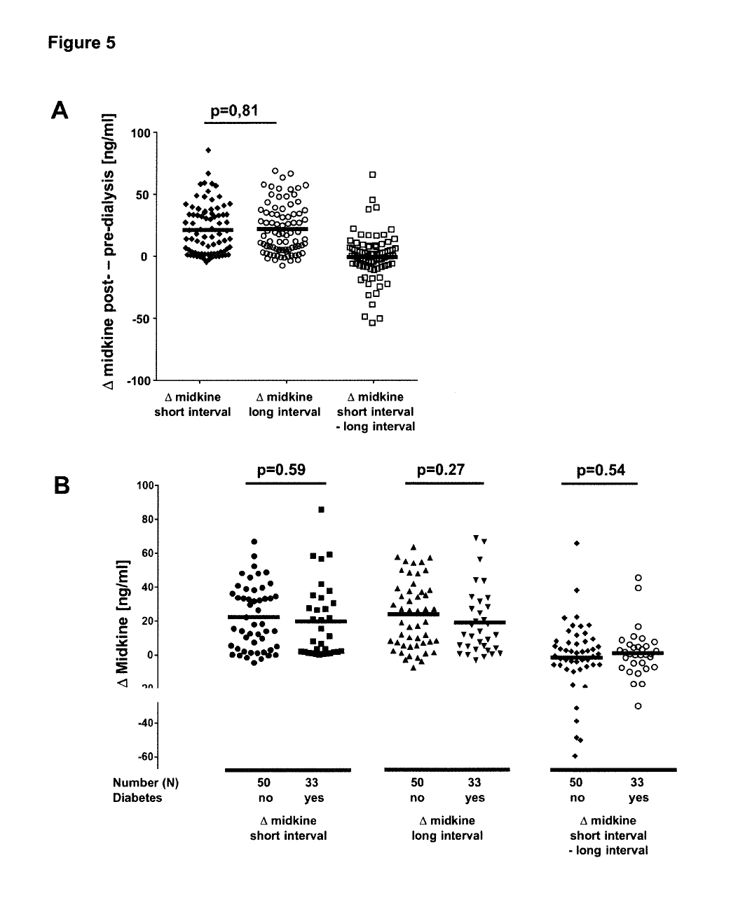

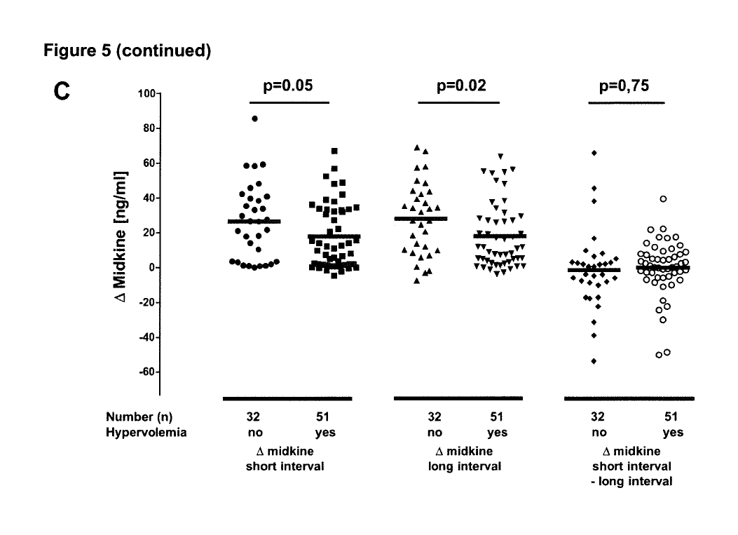

Midkine levels from 100 healthy donors were determined with a mean of 0.59 ng/ml [range 0.02-3.61 ng/ml]. For the 83 dialysis patients following a short (2 day) interval, the mean midkine levels were 2.3±2.3 ng/ml [range 0-10.1 ng/ml] before dialysis and 23.4±19.8 ng/ml [range 0.6-87.1 ng/ml] after dialysis. Following a long (3 day) interval, the mean midkine levels were 3.1±6.9 ng/ml [range 0-58.2 ng/ml] before and 25±19.8 ng/ml [range 0.5-72.3 ng/ml] after dialysis (FIG. 1A). The midkine titers were significantly elevated following hemodialysis compared to the pre-dialysis levels (FIG. 1A). The rise in midkine following hemodialysis was not uniformly observed, given that several patients exhibited lower midkine levels post dialysis (FIG. 1A, FIG. 5A). To visualize the changes, delta (A) midkine values (i.e. midkine after dialysis-before dialysis) were calculated and correlated for both the short and long intervals, yielding a correlation coefficient of r²=0.33 (p<0.001, FIG. 1B).

Midkine Serum Levels are Upregulated by Non-Fractionated, but not Fractionated Heparin.

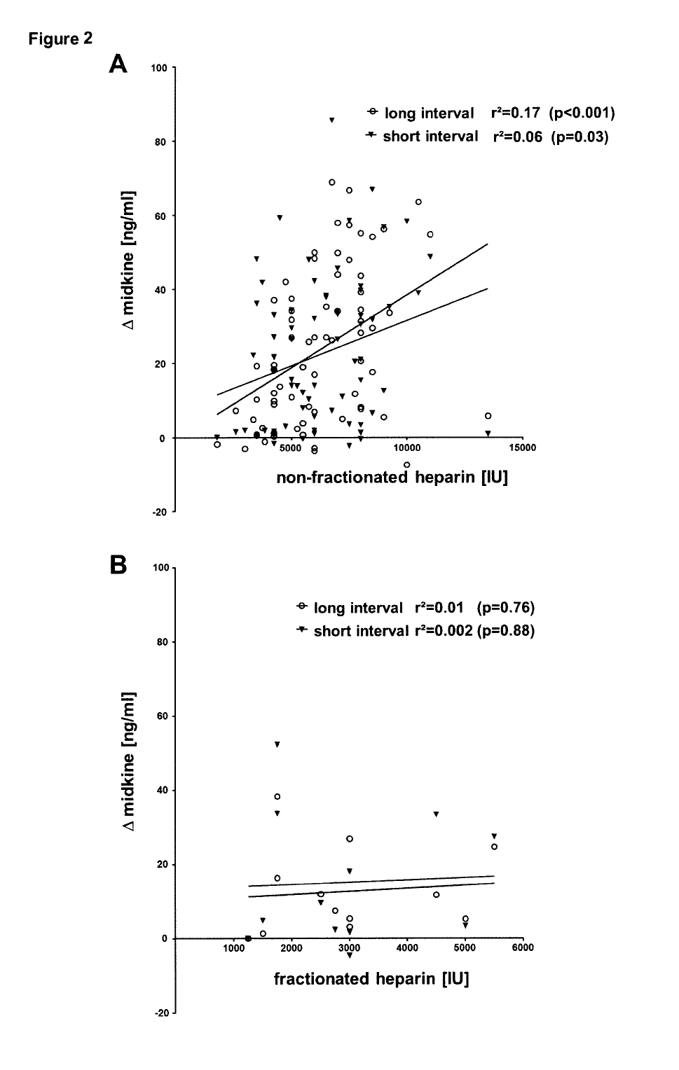

Midkine is also denoted heparin-binding neurotrophic factor. Fujisawa et al. reported that non-fractionated heparin induced endothelial midkine release and thus increased serum levels (Fujisawa K, et al. Increased serum midkine levels during hemodialysis using heparin in chronic renal failure. J Biochem. 1998; 123(5):864-9). In our study, serum midkine levels were compared in patients receiving non-fractionated heparin (n=70) with those receiving fractionated heparin (n=12). Unfractionated heparin was applied following the short (6,222±2,210 IU) and long (6,251±2,180 IU) intervals. The increase in serum midkine levels (expressed as Δmidkine values) during dialysis exhibited a positive correlation with the administered heparin doses following both the short (r²=0.06, p=0.03) and long (r²=−0.17, p<0.001) intervals (FIG. 2A). However the increase in midkine was not uniformly seen; 6/70 patients (short) and 7/70 (long) did not respond.

Fractionated heparin was applied in 12 patients (average 2,958±1,389 IU). The increase of midkine levels was similar after both intervals, 17.6±17 ng/ml and 14.5±11.9 ng/ml, respectively. However, the increase did not positively correlate with the dose of fractionated heparin (FIG. 2B).

Indicators of Systemic Inflammation (CRP, Leukocytosis), A. carotis Intima-Media Thickness, RAAS Inhibitor Intake and Serum Midkine Levels.

Correlation analyses were performed to assess whether serum midkine levels are associated with indicators of systemic inflammation. The assessment of Δmidkine levels in relationship to CRP (short interval p=0.437; long interval p=0.193) and leukocytosis (short interval p=0.166; long interval p=0.068) showed no correlation. Intima-media thickness also did not correlate (data not shown). Studies with mice indicate that midkine synthesis and secretion is regulated by RAAS. Given that 45% of the patients received RAAS inhibitors we analyzed intergroup differences. The comparison revealed that there was no significant difference between both groups.

Serum Midkine Levels in Diabetic Versus Non-Diabetic Patients.

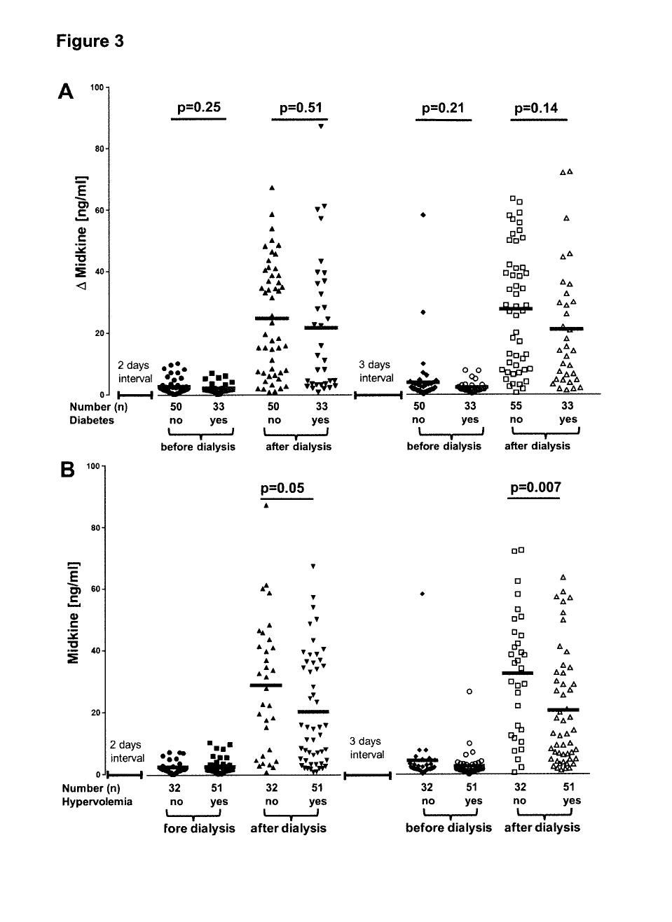

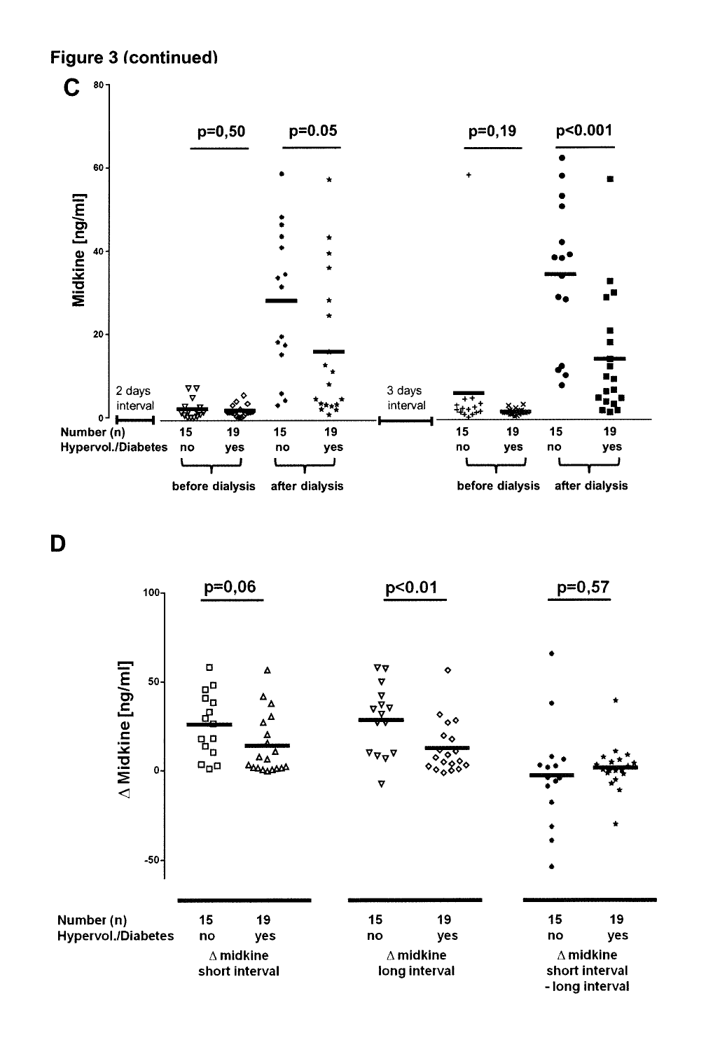

Within the cohort, 33/83 (40%) patients were diagnosed with diabetes mellitus. A statistical analysis for intergroup differences revealed no significant differences (FIG. 3A, FIG. 5B). In diabetics, a trend towards lower midkine levels was observed compared to non-diabetics after the long interval (mean midkine 21±19.8 ng/ml (diabetic) vs. 27.6±19.5 ng/ml (non-diabetic), p=0.14). To assess the variability due to dialysis, ΔΔmidkine values were calculated, which suggest that diabetics tend to be less variable (FIG. 5B).

Serum Midkine Levels and Fluid Volume Status: Clinical Signs of Hypervolemia Inversely Correlate with Midkine Levels.

Hypervolemia was assumed when (i) the actual weight exceeded the clinically defined optimum by >0.5 kg, (ii) the Vena cava diameter was >20 mm wide or (iii) a lung comet tail phenomenon was demonstrated (Table 2A). Accordingly, 51/83 dialysis patients were classified as hypervolemic and 32/83 as normovolemic. Given that reports suggest that vascular changes result in midkine release from endothelial cells within the lung or kidneys (Kosugi T, and Sato W; Midkine and the kidney: health and diseases. Nephrol Dial Transplant. 2012; 27(1):16-21; Netsu S, et al. Midkine exacerbates pressure overload-induced cardiac remodeling. Biochem Biophys Res Commun. 2014; 443(1):205-10; and Jones D R; Measuring midkine: the utility of midkine as a biomarker in cancer and other diseases. Br J Pharmacol. 2014; 171(12):2925-39) we correlated fluid alterations observed inter- and intra-individually with serum midkine changes. Our study protocol included fluid removal of 2.1±1.1 (short) and 2.4±1.1 liters (long). Post-dialysis midkine levels in hypervolemic patients were 20.1±18 ng/ml (short) and 20.4±18.3 ng/ml (long). In normovolemic patients, midkine levels were 28.7±21.5 ng/ml (short) and 32.3±20.1 ng/ml (long), respectively. Hence, the mean midkine levels in hypervolemic patients are significantly lower after dialysis than in patients classified as normovolemic (FIG. 3B, FIG. 5C (A midkine values), short interval p=0.053; long interval p=0.007).

Diabetic Patients with Hypervolemia Versus Non-Diabetics with Normovolemia: Correlation with Dialysis-Related Midkine Response.

19/83 patients (23%) diagnosed with diabetes mellitus were hypervolemic. These constitute a high risk group for cardiovascular disease and high morbidity. For this subgroup mean serum midkine levels after dialysis were 15.8±17.1 ng/ml (short) and 14.0±14.9 ng/ml (long). 18/83 patients (22%) were neither diabetic nor hypervolemic. For this subgroup with a low cardiovascular risk profile, the mean midkine levels were 28.2±18.1 ng/ml (short) and 33.7±18.6 ng/ml (long) (FIG. 3C). Comparing midkine levels and Δmidkine values for these subgroups with differing cardiovascular risk profiles provided significantly lower midkine levels in the hypervolemic diabetics after cardiovascular stress (FIGS. 3C and 3D, respectively, long interval p<0.001). This was confirmed in a separate cohort (3 days interval p<0.002). The calculated AUC by receiver operator characteristics was 0.709 for the 3 days interval and 0.796 (sensitivity 80%, specificity 79.9%) for both intervals combined.

(Cardiovascular) Mortality and Midkine Levels.

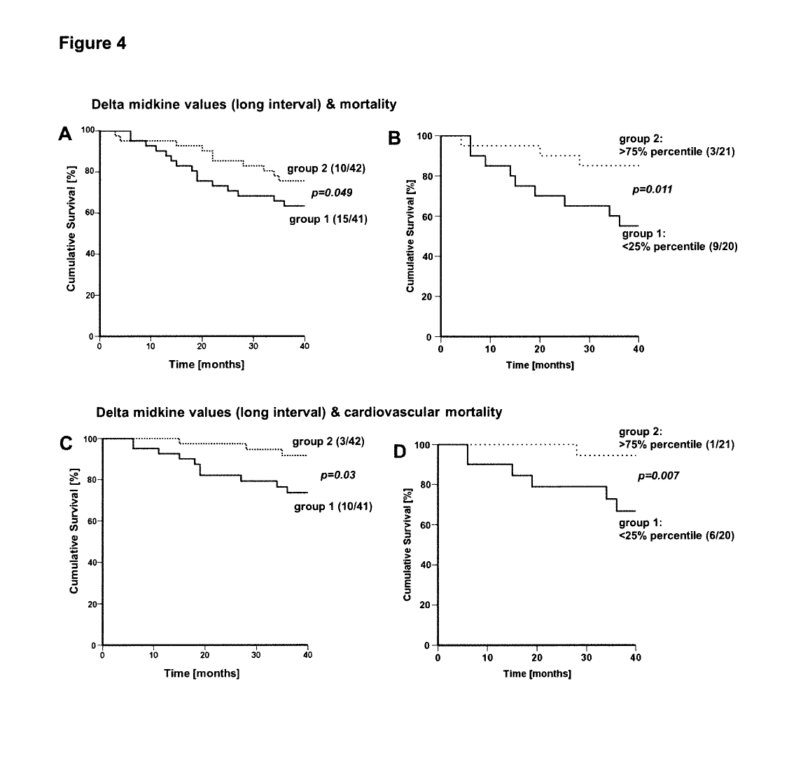

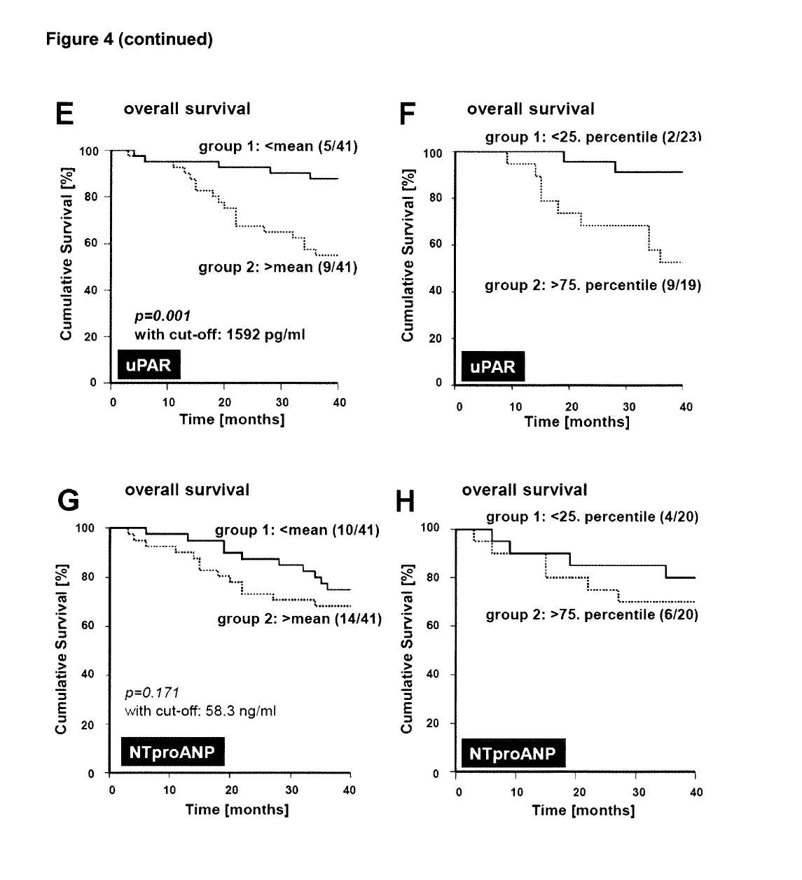

Sample collection began in 06/2012 and patients were followed-up for 36 months. By 06/2015, 25/83 (30%) of hemodialysis patients had died, 13 (16%) due to cardiovascular events (myocardial infarction, strokes). At the time of recruitment, the deceased patients had significantly lower midkine levels after hemodialysis (short interval: 20.7±17.9 ng/ml; long interval 18.6±15.8 ng/ml) compared to the living patients (short interval: 24.6±20.5 ng/ml; long interval: 27.7±20.8 ng/ml). A classification of midkine levels below/above the mean for all dialysis patients (n=83) and subgrouping revealed that low midkine levels after hemodialysis constitutes a significant adverse prognosis marker for overall (FIG. 4A, p=0.049) and cardiovascular mortality (FIG. 4B, p=0.03). For the latter group, the mean post-dialysis midkine levels were 16.8±15.7 ng/ml (short) and 13.4±13.6 ng/ml (long).

Biomarkers of Endothelial Dysfunction, (Cardiovascular) Mortality and Midkine to Predict Adverse (Cardiovascular) Outcome.

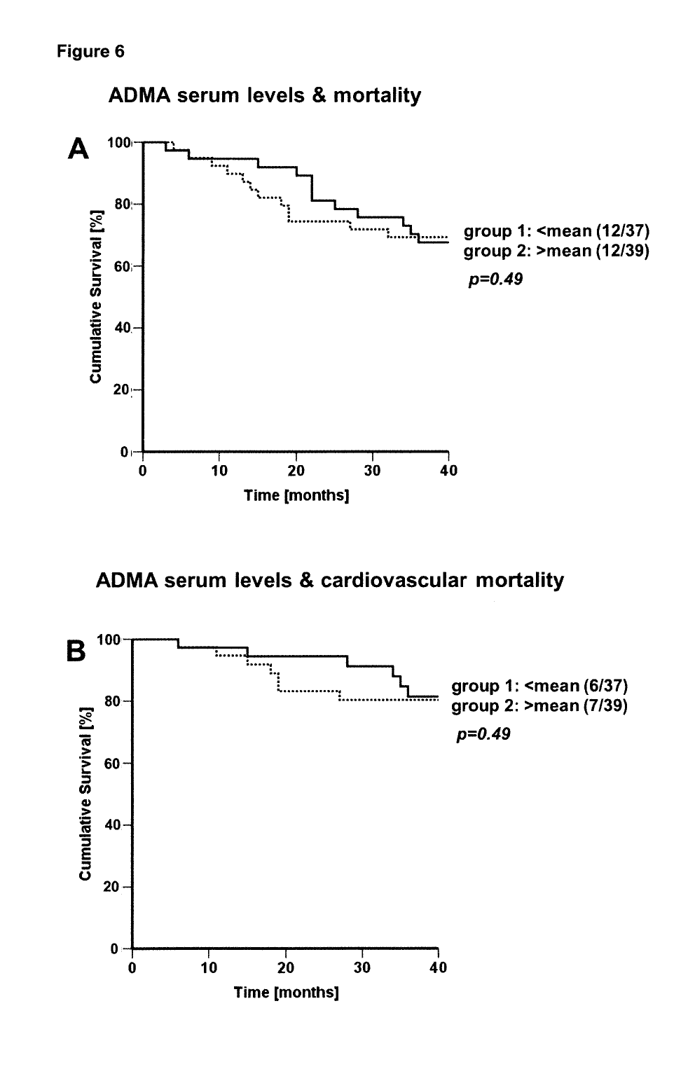

Sample collection began in 06/2012 and patients were followed-up for 48 months. By 06/2015, 30% (25/83) of hemodialysis patients had died, 13 (16%) due to cardiovascular events (sudden cardiac death, myocardial infarction, strokes). For 81 patients, the plasma levels of ADMA, SDMA, and L arginine in pre-dialysis samples were determined. For those patients Kaplan-Meier survival curves were plotted with ADMA values above/below average, exhibiting no significant differences in (cardiovascular) mortality over 36 months (FIG. 6). Furthermore, average ADMA, SDMA, and L arginine serum values were determined for patients with Δmidkine values above/below average, again without significant differences (not shown), suggesting that midkine release and basal ADMA, SDMA, and L-arginine metabolism are not interconnected (Table 2B). At the time of recruitment, the deceased patients had significantly lower midkine values after hemodialysis (2 days interval: 20.7±17.9 ng/ml; 3 days interval 18.6±15.8 ng/ml) compared to the living patients (2 days interval: 24.6±20.5 ng/ml; 3 days interval: 27.7±20.8 ng/ml). A classification of midkine values below/above the mean for all dialysis patients (n=83) and subgrouping revealed that low midkine levels after hemodialysis constitutes a significant adverse prognosis marker for overall (FIG. 6A, p=0.04) and cardiovascular mortality (FIG. 6B, p=0.03). For the latter group, the mean post-dialysis midkine values were 16.8±15.7 ng/ml (2 days interval) and 13.4±13.6 ng/ml (3 days interval). With cut-off delta midkine values defined at the 25 and 75 percentiles the levels of significance reached a p-value of <0.01 and <0.007 (FIGS. 4A through D). Further biomarkers uPAR and NTproANP were tested with the same samples. The results confirm the predictive value of uPAR. Notably, NTproANP as marker for cardiac stress was of inferior predictive value for overall and cardiovascular survival in the dialysis cohort (FIGS. 4E through H; Table 3). Notably subanalyses revealed that there is a correlation of NTproANP and absolute midkine values in serum samples of diabetic dialysis patients (r=0.6) whereas such a correlative link is much weaker between midkine and uPAR. Finally a heat map was created (not shown) to determine the link between the different serum markers. The cluster analysis was performed by Genesis software. A “below than average” increase of midkine was linked with elevated uPAR serum levels and poor outcome. In contrast tenascin-C, galectin and MTproANP are distinct and nevertheless help to distinguish patient subgroups that are generated by clustering.

Discussion

Midkine is discussed as biomarker for diverse diseases, especially within the context of cancer, cardiovascular and kidney diseases (Jones D R. Measuring midkine: the utility of midkine as a biomarker in cancer and other diseases. Br J Pharmacol. 2014; 171(12):2925-39). Serum midkine levels in healthy subjects are mostly determined within a narrow range that depends on the applied detection system. Beyond these “background” levels, several diseases are known to be accompanied by elevated midkine serum levels, e.g. cancers of different origin, ischemia of brain, limbs or kidneys (Muramatsu T; Midkine, a heparin-binding cytokine with multiple roles in development, repair and diseases. Proc Jpn Acad Ser B Phys Biol Sci. 2010; 86(4):410-25) and congestive heart failure (Kitahara T, et al. Serum midkine as a predictor of cardiac events in patients with chronic heart failure. J Card Fail. 2010; 16(4):308-13). Elevated midkine levels are mostly interpreted as being indicative of presence of disease, rapid “normalization” following tumor eradication within short periods of time has been observed (Krzystek-Korpacka M, et al.; Circulating midkine in malignant and non-malignant colorectal diseases. Cytokine. 2013; 64(1):158-64). Compared to the analyzed healthy control cohort midkine baseline levels are four-fold higher in the dialysis cohort. This is in line with two-fold higher midkine serum levels in patients with congestive heart disease (Kitahara T, et al.; Serum midkine as a predictor of cardiac events in patients with chronic heart failure. J Card Fail. 2010; 16(4):308-13), a condition often diagnosed in dialysis patients.

The main challenge to the use of midkine as diagnostic biomarker by single determinations is the generality of its regulation and lack of specificity for particular diseases (ones DR. Measuring midkine: the utility of midkine as a biomarker in cancer and other diseases. Br J Pharmacol. 2014; 171(12):2925-39). To address this issue and not rely on single serum levels determined in index patients the present study protocol comprised of serial midkine determinations, performed before and after dialysis sessions, repeated on two occasions and with differing fluid retention. The primary hypothesis was that midkine release in dialysis patients is altered due to the dialysis procedure per se with preexisting generalized endothelial cell damage due to an inflammatory milieu and neoangiogenesis in dialysis patients. A positive fluid balance resulting in signs of hypervolemia is a common finding in CKD. Fluid overload is associated with arterial hypertension, left ventricular hypertrophy, and accelerated arteriosclerosis. The dialysis procedure itself with fluid removal constitutes a high cardiovascular stress, that exceeds standard diagnostic adenosine stress testing or exercise electrocardiography (Assa S, et al. Comparison of cardiac positron emission tomography perfusion defects during stress induced by hemodialysis versus adenosine. Am J Kidney Dis. 2012; 59(6):862-4). Therefore, maintaining an euvolemic state is important for preventing cardiovascular complications (Konings C J, et al. Fluid status, blood pressure, and cardiovascular abnormalities in patients on peritoneal dialysis. Perit Dial Int. 2002; 22(4):477-87). Fluid has to be mobilized by the body within a short period of dialysis time and prolonged redistribution takes place especially within the interdialytic intervals. Herein, 63% of the patients were classified as hypervolemic and we found a strong negative correlation between hypervolemia and mean midkine levels post dialysis. When the mean midkine level post dialysis was lower than average a fluid overload was more likely present, suggesting a direct link between fluid balance and midkine release. In patients with clinical signs of hypervolemia midkine levels rose by about 6 to 8.9-fold, whereas in the absence of hypervolemia this rise was >12.2 to 14.6-fold. Thus, vascular stress caused by chronic hypervolemia may suppress midkine release by endothelial cells or, conversely, a diminished response and rise of serum midkine levels may be causatively linked with a failure to mobilize interstitial fluid.

The sources of midkine and mechanisms of release are not well understood (Salaru D L, and Mertens P R; Lessons from the heart and ischemic limbs: midkine as anti-inflammatory mediator for kidney diseases? Int Urol Nephrol. 2013; 45(3):893-7). The lung endothelium may constitute a major source, given that a vicious cycle of positive feedback in the midkine-angiotensin II pathway with oxidative stress and subsequently up-regulated ACE exists (Sato W, Sato Y; Midkine in nephrogenesis, hypertension and kidney diseases. Br J Pharmacol. 2014; 171(4):879-87). Kidney-lung interactions might partly account for the pathogenesis of hypertension and kidney damage. However, these have not been analyzed in detail in dialysis patients where kidney fibrosis will mostly prevail. In a single study a stimulatory effect of heparin application on serum midkine release in dialysis patients and healthy controls was determined (Fujisawa K, et al. Increased serum midkine levels during hemodialysis using heparin in chronic renal failure. J Biochem. 1998; 123(5):864-9), whereas other studies quantified midkine and inflammatory cytokine release in sepsis and vascular disease (Aydemir B, et al. The Circulating Levels of Selenium, Zinc, Midkine, Some Inflammatory Cytokines, and Angiogenic Factors in Mitral Chordae Tendineae Rupture. Biol Trace Elem Res. 2015; and Salaru D L, et al. Serum levels for midkine, a heparin-binding growth factor, inversely correlate with angiotensin and endothelin receptor autoantibody titers in patients with macroangiopathy. Int Angiol. 2014; 33(4):372-378). In the present study a differential response to heparin was observed. The dose of non-fractionated heparin correlates with the rise in midkine (r²=0.33), whereas fractionated heparin does not. This may be due to structural differences between the two (Rabenstein D L. Heparin and heparan sulfate: structure and function. Nat Prod Rep. 2002; 19(3):312-331). It is likely that non-fractionated heparin, which is longer and has more variable binding sites, enhances midkine expression, release or prolongs its half-life.

The most striking finding of this study is the predictive value of midkine release after cardiovascular stress for (cardiovascular) mortality in patients. When the rise of serum midkine levels during cardiovascular stress was below average, the likelihood of (cardiovascular) mortality was significantly increased; a finding that becomes even more apparent when the upper 75 and lower 25 percentiles are compared (FIG. 4C). Thus, following a long dialysis-free interval testing for midkine levels before and after dialysis are suitable for risk stratification. Clearly, this testing outperforms other markers for endothelial dysfunction, such as ADMA, which are controversially discussed as predictors of mortality (Frenay A R, et al.; Plasma ADMA associates with all-cause mortality in renal transplant recipients. Amino Acids. 2015; 47(9):1941-1949; and Vlahakos D V, et al.; 1c.09: Serum Resistin as an Independent Biomarker Associated with All-Cause and Cardiovascular Mortality in Elderly Hypertensive, Non-Diabetic Patients with Chronic Kidney Disease (Ckd). J Hypertens. 2015; 33 Suppl 1:e11-2). Furthermore, one might question how serum midkine levels can be therapeutically modified and whether midkine is the missing link to explain fluid retention (Floege J, and Uhlig S; Kidney calling lung and call back: how organs talk to each other. Nephrol Dial Transplant. 2010; 25(1):32-34). The latter is of paramount importance for dialysis patients, where non-adherence to “fluid recommendations” is daily practice and a less-than-appropriate midkine release may possibly explain fluid retention and hyperhydration. Recombinant human midkine is available and enters clinical studies (Murasugi A; Efficient expression and purification of recombinant therapeutic protein candidates, human midkine and pleiotrophin. Curr Pharm Biotechnol. 2013; 14(8):768-84), e.g. to combat heart failure. Previous studies suggest that midkine regulates RAAS (Salaru D L, Mertens P R. Lessons from the heart and ischemic limbs: midkine as anti-inflammatory mediator for kidney diseases? Int Urol Nephrol. 2013; 45(3):893-897), conversely, its levels are not affected by RAAS inhibition in the present study.

### CONCLUSIONS

Midkine has many biological activities. In end-stage renal disease, baseline levels are elevated, a midkine release with 6- to 15-fold higher serum levels is incited by dialysis procedure. Several confounders and effectors are linked with the magnitude of the midkine release: non-fractionated heparin dose-dependently stimulates a midkine release, whereas hypervolemia and diabetes are associated with lower midkine release and serum levels following cardiovascular stress. The (cardiovascular) mortality is significantly higher for patients with less-than-average midkine release during hemodialysis. These findings show a key protective role for the heparin-binding growth and differentiation factor midkine in fluid homeostasis.

TABLE 1A

| | | |
|---|---|---|
| Mean age [years] | | 64 ± 16 |
| On dialysis since [years] | | 4.1 ± 3.9 |
| Sex | Male | 54/83 (65%) |
| | Female | 29/83 (35%) |
| Co-morbidities | Arterial hypertension | 82/83 (99%) |
| | Coronary artery disease | 40/83 (48%) |
| | Diabetes mellitus | 33/83 (40%) |
| | Carcinoma | 4/83 (5%) |
| | Cerebrovascular disease | 17/83 (21%) |
| Anticoagulation | | |
| Non-fractionated heparin: | Short interval [IU] | 6.222 ± 2.210 |
| 70/82 (84%) | Long interval [IU] | 6.251 ± 2.180 |
| Fractionated heparin [IU]: | Short/long interval [IU] | 2.958 ± 2.875 |
| 12/82 (15%) | | |
| Weight difference before/ | | |
| after dialysis | | |
| | Short interval [kg] | 2.1 ± 1.1 |
| | Long interval [kg] | 2.4 ± 1.1 |
| Hypervolemia | | 51/83 (61%) |
| Intima media thickness [mm] | | 0.9 ± 0.2 |

TABLE 1B

| | | | |
|---|---|---|---|
| CrP (range) [mg/dl] | | 1.4 ± 6.5 | (0-59) |
| Leucocytes (range) [Gpt/1] | | 6.4 ± 2.1 | (2.4-14.5) |
| Calcium (range) [mmol/1] | | 2.2 ± 0.2 | (1.8-2.5) |
| Phosphate (range) [mmol/1] | | 1.6 ± 0.5 | (0.9-2.9) |
| Midkine (range) [ng/ml] | | | |
| Short interval: (range) | Before dialysis | 2.3 ± 2.3 | (0-10.1) |
| | After dialysis | 23.4 ± 19.8 | (0.6-87.1) |
| Long interval: (range) | Before dialysis | 3.1 ± 6.9 | (0-58.2) |
| | After dialysis | 245 ± 19.8 | (0.5-72.3) |

TABLE 2A
Fluid homeostasis assessment

| | Hypervolemia defined by | | |
|---|---|---|---|
| | | Elevated | |
| | Comet tail | weight | V. cava diam- |
| | sign lungs | (+>0.5 kg) | eter > 20 mm |
| Number of patients (n) | 11 | 38 | 22 |

TABLE 2B
ADMA, SDMA, L-arginine and delta midkine serum levels

| | Long interval | Long interval | |
|---|---|---|---|
| | Above mean | below mean | |
| | (24.9 ng/ml) | (24.9 ng/ml) | |
| Delta Midkine | n = 42 | n = 41 | p-value |
| ADMA [μmol/l] | 0.49 ± 0.99 | 0.49 ± 0.09 | 0.88 |
| SDMA [μmol/l] | 1.28 ± 0.36 | 1.33 ± 0.1 | 0.65 |
| L-arginine [μmol/l] | 22.78 ± 9.4 | 24.11 ± 20.7 | 0.72 |

TABLE 3A

| | | | | Sensitiv- | Specific- |
|---|---|---|---|---|---|
| 3 year survival | Cut-off | | AUC | ity (%) | ity (%) |
| uPAR | >1260 | pg/ml | 0.713 | 77 | 67 |
| Δmidkine 3 days interval | <27 | ng/ml | 0.618 | 76 | 43 |
| NTproANP | >61 | ng/ml | 0.617 | 30.7 | 91 |

TABLE 3B

| | | | | Sensitiv- | Specific- |
|---|---|---|---|---|---|
| 4 year survival | Cut-off | | AUC | ity (%) | ity (%) |
| uPAR | >1224 | pg/ml | 0.736 | 72 | 70 |
| Δmidkine 3 days interval | <27 | ng/ml | 0.641 | 75 | 50 |
| NTproANP | >59 | ng/ml | 0.605 | 31 | 90 |

TABLE 3C
AUC for prediction of survival at 3 years

| | | Δmidkine 3 days | |
|---|---|---|---|
| Combination of | uPAR | interval | NTproANP |
| uPAR | 0.713 | 0.713 | 0.710 |
| Δmidkine 3 days interval | | 0.618 | 0.650 |
| NTproANP | | | 0.617 |

TABLE 3D
AUC for prediction of survival at 4 years

| | | Δmidkine 3 days | |
|---|---|---|---|
| Combination of | uPAR | interval | NTproANP |
| uPAR | 0.736 | 0.754 | 0.736 |
| Δmidkine 3 days interval | | 0.641 | 0.646 |
| NTproANP | | | 0.605 |

1. A method for the prognosis of adverse outcomes in a subject comprising the following steps:
determining the midkine level in a sample of said subject; and
comparing the determined midkine level to a control midkine level;
wherein said sample is taken after the subject has been subjected to cardiovascular stress;
wherein said control midkine level is derived from control sample(s) from one or more subjects not showing adverse outcomes, wherein the control sample(s) have been taken after said one or more subjects not showing adverse outcomes have been subjected to cardiovascular stress; and
wherein a decreased determined midkine level as compared to the control midkine level is indicative of an adverse outcome.

2. The method according to claim 1, wherein the adverse outcome is selected from the group consisting of hypervolemia, cardiovascular mortality, mortality, myocardial infarction, stroke, and congestive heart failure; preferably a decreased midkine level is indicative of an adverse outcome within 36 months.

3. The method according to claim 1, wherein said cardiovascular stress is selected from the group consisting of dialysis, hemodialysis, stress electrocardiogram, cardiac stress, and cardiac stress testing, cardiac stress testing using stimulatory drugs, preferably adenosine, and dobutamine.

4. The method according to claim 1, wherein the determination of the Δmidkine value is started following a cardiovascular stress free interval of 2 days or of 3 days.

5. The method according to claim 1, wherein said midkine control level is 33 ng/ml, preferably 30 ng/ml, more preferably 28 ng/ml.

6. The method according to claim 1, wherein the adverse outcome is mortality, and wherein said midkine control level is 27 ng/ml, preferably 24 ng/ml even more preferably 20 ng/ml.

7. The method according to claim 1, wherein the adverse outcome is cardiovascular mortality, and wherein said midkine control level is 18 ng/ml.

8. The method according to claim 1, wherein the presence of a cardiosvascular disease or diabetes in said subject to be diagnosed further indicates an adverse outcome.

9. A method for the prognosis of adverse outcomes in a subject comprising the following steps:
determining in samples of said subject the increase in midkine levels (Δmidkine value) during cardiovascular stress;
comparing the determined Δmidkine value to a control Δmidkine value;
wherein said control Δmidkine value is derived from one or more subjects not showing adverse outcomes;
wherein a decreased determined Δmidkine value as compared to the control Δmidkine value is indicative of an adverse outcome.

10. A method for the prognosis of adverse outcomes in a subject undergoing dialysis therapy comprising the step of:
determining in samples of said subject the increase in midkine levels during dialysis;
wherein an increase in midkine levels in samples of said subject during dialysis of less than 10 fold is indicative of an adverse outcome; preferably of less than 8 fold, more preferably of less than 7 fold.

11. A method for the prognosis of adverse outcomes in a subject undergoing dialysis therapy comprising the step of:
determining in samples of said subject the increase in midkine levels during dialysis (Δmidkine value);
wherein a Δmidkine value of less than 25 ng/ml is indicative of the prognosis for an adverse outcome.

12. The method according to claim 9, wherein the determination of the Δmidkine value comprises the following steps:
determining the level of midkine in a sample taken before the subject is subjected to a cardiovascular stress;
determining the level of midkine in a sample taken after the subject has been subjected to a cardiovascular stress;
calculating the difference between the two determined midkine levels and thereby obtaining the Δmidkine value.

13. The method according to claim 1, wherein the sample is selected from the group consisting of urine sample, blood sample, serum sample, and plasma sample; preferably serum sample.

14. The method according to claim 1, wherein the samples are either processed immediately or stored at temperatures of −20° C. or less.

15. The method according to claim 1, wherein the levels of midkine are detected in an immunoassay.

16. The method according to claim 15, wherein the immunoassay is selected from the group of immunoprecipitation, enzyme immunoassay (EIA), radioimmunoassay (RIA), enzyme-linked immunosorbent assay (ELISA), fluorescent immunoassay, a chemiluminescent assay, an agglutination assay, nephelometric assay, turbidimetric assay, a Western Blot, a competitive immunoassay, a noncompetitive immunoassay, a homogeneous immunoassay a heterogeneous immunoassay, a bioassay and a reporter assay such as a luciferase assay or luminex.

17. The method according to claim 1, comprising the steps of
(a) contacting the sample with a midkine antibody or an antigen binding fragment thereof under conditions allowing for the formation of a complex between said midkine antibody or the antigen binding fragment thereof with the midkine; and
(b) detecting the formed complex.

18. The method of claim 17, wherein the midkine antibody or the antigen binding fragment thereof is immobilized on a surface.

19. The method according to claim 17, wherein the complex is detected using a secondary antibody against midkine.

20. The method according to claim 19, wherein the secondary antibody is labelled with a detectable marker.

21. Use of a midkine antibody or an antigen binding fragment thereof for the prognosis of adverse outcomes in a subject undergoing dialysis therapy.

22. Midkine for use in the treatment or the prevention of an adverse outcome, wherein midkine is administered to the subject, if the subject exhibits midkine levels in a sample taken after the subject being subjected to a cardiovascular stress indicative of an adverse outcome or if the subject exhibits an increase of midkine levels during cardiovascular stress (Δmidkine values) being indicative of an adverse outcome.

23. Midkine for use in the treatment or the prevention of an adverse outcome, wherein the subject has been prognosed for an adverse outcome in a method according to claim 1.
